SEQUENCE LISTING (17 sequences)

SEQ ID NO: 1 - DNA, 2133 bp, Trypanosoma cruzi
tgttccccttttctcttcccaactttctccggcggcaatccccctgcaaagagacgatcttgacaccattgttttaggcataatagaagttctacaaacaacgcccgaaggacacacaggcaggcaccgactaccatggggaaaacagtcgttgtggccagtaggatgttctggctaatgtttttcgtgccgcttcttcttgcgatctgccccagcgagcccgcgtacgccttggcacccggatcgagccgagttgagctgtttaagcgtaagaattcgacggtgccgtttgaagacaaggccggcaaagtcaccgagcgggttgtccactcgttccgcctccccgcccttgttaatgtggacggggtgatggttgccatcgcggacgctcgctacgacacatccaatgacaactccctcattgatacggtggcgaagtacagcgtggacgatggggagacgtgggagacccaaattgccatcaagaacagccgtgtatcgtctgtttctcgtgtggtggatcccaccgtgattgtgaagggcaacaagctttacgtcctggttggaagctactatagttcgagaagctactggtcgtcgcatggtgatgcgagagactgggatattctgcttgccgttggtgaggtcacgaagtccactgcgggcggcaagataactgcgagtatcaaatgggggagccccgtgtcactgaagaagttttttccggcagaaatggaaggcatgcacacaaatcaatttcttggcggcgcgggtgttgccattgtagcgtccaacgggaatcttgtgtaccctgtgcaggttacgaacaaaaagaagcaagttttctccaagatcttctactcggaagatgatggcaagacgtggaagtttgggaagggtaggagcgattttggctgctctgaacctgtggcccttgagtgggaggggaagctcatcataaacacccgagttgactggaaacgccgtctggtgtacgagtccagtgacatggagaaaccgtgggtggaggctgtcggaaccgtctcgcgtgtgtggggcccctcaccaaaatcgaaccagcccggcagtcagagcagcttcactgccgtgaccatcgaaggaatgcgtgtgatgctcttcacacacccgctgaattttaagggaaggtggctgcgcgaccgactgaacctctggctgacggataaccagcgcatttataacgttgggcaagtatccattggtgatgaaaattccgcctacagctccgtcctgtacaaggatgataagctgtactgtttgcatgagatcaacacggacgaggtgtacagccttgtttttgcacgcctggttggcgagctacggatcattaaatcagtgctgcggtcctggaagaattgggacagccacctgtccagcatttgcacccctgctgatccagccgcttcgtcgtcagagagtggttgtggtcccgctgtcaccacggttggtcttgttggctttttgtccggcaacgcctcccaaaacgtatgggaggatgcgtaccgctgcgtcaacgcaagcacggcaaatgcggagagggttcggaacggtttgaagtttgcgggggttggcggaggagcgctttggccggtgagccagcaggggcagaatcagcggtatcgttttgcaaaccacgcgttcacgctggtggcgtcggtgacgattcacgaggctccgagggccgcgagtcccttgctgggtgcgagcctggactcttctggcggcaaaaaactcctggggctctcgtacgacgagaagcaccagtggcagccaatatacggatcaacgccggtgacgccgacgggatcgtgggagacgggtaaaaggtaccacttggttcttacgatggcgaataaaattggctccgtgtacattgatggagaacttctggagggttcaggacagaccgttgtgccagacgggaggacgcctgacatctcccacttctacgttggcgggtataaaaggagtgatatgccaaccataagccacgtgacggtgaataatgttcttctttacaaccgacagctgaataccgaggagatcaggaccttgttcttgagccaggaccttattggcacggaagcacacatg

SEQ ID NO: 2 - protein, 666 aa, Trypanosoma cruzi
MGKTVVVASRMFWLMFFVPLLLAICPSEPAYALAPGSSRVELFKRKNSTVPFEDKAGKVTERVVHSFRLPALVNVDGVMVAIADARYDTSNDNSLIDTVAKYSVDDGETWETQIAIKNSRVSSVSRVVDPTVIVKGNKLYVLVGSYYSSRSYWSSHGDARDWDILLAVGEVTKSTAGGKITASIKWGSPVSLKKFFPAEMEGMHTNQFLGGAGVAIVASNGNLVYPVQVTNKKKQVFSKIFYSEDDGKTWKFGKGRSDFGCSEPVALEWEGKLIINTRVDWKRRLVYESSDMEKPWVEAVGTVSRVWGPSPKSNQPGSQSSFTAVTIEGMRVMLFTHPLNFKGRWLRDRLNLWLTDNQRIYNVGQVSIGDENSAYSSVLYKDDKLYCLHEINTDEVYSLVFARLVGELRIIKSVLRSWKNWDSHLSSICTPADPAASSSESGCGPAVTTVGLVGFLSGNASQNVWEDAYRCVNASTANAERVRNGLKFAGVGGGALWPVSQQGQNQRYRFANHAFTLVASVTIHEAPRAASPLLGASLDSSGGKKLLGLSYDEKHQWQPIYGSTPVTPTGSWETGKRYHLVLTMANKIGSVYIDGELLEGSGQTVVPDGRTPDISHFYVGGYKRSDMPTISHVTVNNVLLYNRQLNTEEIRTLFLSQDLIGTEAHM

SEQ ID NO: 3 - DNA, 5403 bp, Trypanosoma cruzi
aaagaccgttggaagaagaaagaaggttccggagcgtggccaccaccaacgatgaactgccacaattgcgtgctgtccgcgggcggtacccggcgctttgagcccacggcgacttgtgtgttcccctttctcttcccactttctccgcggcaatccccctgcaaagagacgatcttgacaccattgttttaggcataatagaagttctacaaacaacgcccgaaggacacacaggcaggcaccgactacgatggggaaaacagtcgttgtggccagtaggatgttctggctaatgtttttcgtgccgcttcttcttgcgatctgccccagcgagcccgcgtacgccctggcacccggatcgagccgagttgagggtttaagcgtaagaattcgacggtgccgtttgaagacaaggccggcaaagtcaccgagcgggttgtccactcgttccgcttccccgcccttgttaatgtggacggggtgatggttgccatcgcggacgctcgctacgaaacatccagtgaaaactccctcattgatacggtggcgaagtacagcgtggacgatggggagacgtgggagacccaaattgccatcaagaacagccgtgtatcgtctgtttctcgtgtggtggatcccaccgtgattgtgaagggcaacaagctttacgtcctggttggaagctactatagttcgagaagctactggtcgtcgcatggtgatgcgagagactgggatattctgcttgccgttggtgaggtcacgaagtccactgcgggcggcaagataactgcgagtatcaaatgggggagccccgtgtcactgaagaagttttttccggcagaaatggaaggcatgcacacaaatcaatttcttggcggcgcgggtgttgccattgtagcgtccaacgggaatcttgtgtaccctgtgcaggttacgaacaaaaggaagcaagttttctccaagatcttctactcggaagatgatggcaagacgtggaagtttgggaagggtaggagcgattttggctgctctgaacctgtggcccttgagtgggaggggaagctcatcataaacacccgagttgactggaaacgccgtctggtgtacgagtccagtgacatggagaaaccgtgggtggaggctgtcggaaccgtctcgcgtgtgtggggcccctcaccaaaatcgaaccagcccggcagtcagacgagcttcactgccgtgaccatcgaaggaatgcgtgtgatgctcttcacacacccgctgaattttaagggaaggtgcgtgcgcgaccgactgaacctctggctgacggataaccagcgcatttataacgttgggcaagtatccattggtgatgaaaattccgcctacagctccgtcctgtacaaggatgataagctgtactgtttgcatgagatcaacacggacgaggtgtacagccttgtttttgcacgcctggttggcgagctacggatcattaaatcagtgctgcggtcctggaagaattggacagccacctgtccagcatttgcacccctgctgatccagccgcttcgtcgtcagagagtggttgtggtcccgctgtcaccacggttggtcttgttggctttttgtcggcaacgcctcccaaaacgtatgggaggatcgtaccgctgcgtcaacgcaagcacggcaaatgcggagagggttcggaacggtttgaagtttgcgggggttggcggaggagcgctttggccggtgagccagcaggggcagaatcagcggtatcgttttgcaaaccacgcgttcacgctggtggcgtcggtgacgattcacgaggctccgagggccgcgagtcccttgctgggtgcgagcctggactcttctggcggcaaaaaactcctggggctctcgtacgacgagaagcaccagtggcagccaatatacggatcaacgccggtgacgccgacgggatcgtgggagacgggtaaaaggtaccacttggttcttacgatggcgaataaaattggctccgtgtacattgatggagaacttctggagggttcaggacagaccgttgtgccagacgggaggacgcctgacatctcccacttctacgttggcgggtataaaaggagtgatatgccaaccataagccacgtgacggtgaataatgttcttctttacaaccgacgacagctgaataccgaggagatcaggaccttgttcttgagccaggaccttattggcacggaagcacacatggacagcagcagcgacagcagtgcccacagtacgccctcaactcccgctgacagcagtgcccacagtacgccctcaactcccgttgacagcagtgcccacagtacgccctcgactcccgctgacagcagtgcccacggtacgccctcaactcccgttgacagcagtgcccacggtacgccctcaactcccgctgacagcagtgcccacggtacgccctcaactcccgttgacagcagtgcccacagtacgccctcaactcccgttgacagcagtgcccacagtacgccctcaactcccgttgacagcagtgcccacggtgcgccctcaactcccgctgacagcagtgcccacggtacgccctcgactcccgttgacagcagtgcccacggtacgccctcgactcccgctgacagcagtgcccacagtacgccctcgactcccgctgacagcagtgcccacagtacgccctcgactcccgctgacagcagtgcccacagtacgccctcgactcccgttgacagcagtgcccacggtacgccctcgactcccgctgacagcagtgcccacagtacgccctcgactcccgctgacagcagtgcccacggtacgccctcaactcccgttgacagcagtgcccacagtacgccctcgactcccgttgacagcagtgcccacggtacgccctcaactcccgttgacagcagtgcccacagtacgccctcgactcccgttgacagcagtgcccacggtacgccctcaactcccgttgacagcagtgcccacagtacgccctcgactcccgctgacagcagtgcccacagtacgccctcaactcccgctgacagcagtgcccacggtacgccctcaactcccgttgacagcagtgcccacagtacgccctcgactcccgctgacagcagtgcccacagtacgccctcaactcccgttgacagcagtgcccacagtacgccctcaactcccgctgacagcagtgcccacggtacgccctcaactcccgttgacagcagtgcccacggtacgccctcgactcccgctgacagcagtgcccacagtacgccctcgactcccgctgacagcagtgcccacagtacgccctcgactcccgctgacagcagtgcccacagtacgccctcaactcccgttgacagcagtgcccacagtacgccctcaactcccgctgacagcagtgcccacagtacgccctcaactcccgctgacagcagtgcccacagtacgccctcgactcccgctgacagcagtgcccacagtacgccctcaactcccgttgacagcagtgcccacagtacgccctcaactcccgctgacagcagtgcccacggtacgccctcgactcccgctgacagcagtgcccacagtacgccctcgactcccgttgacagcagtgcccacagtacgccctcgactcccgctgacagcagtgcccacggtacgccctcgactcccgctgacagcagtgcccacagtacgccctcgactcccgctgacagcagtgcccacggtacgccctcgactcccgctgacagcagtgcccacagtacgccctcaactcccgctggcagcagcgccaatggtacggttctgattttgcccgatggcgctgcactttcgaccttttcgggcggagggcttcttctgtgtgcgtgtgctttgctgctgcacgtgttttttatggcagttttctgatgtagtgagagagtctcctaacaaatgtagataaattcataattgtggtgtgaaccgtttgggtaaatgtgtgtgtgcgctctcataaggaaatgatttccagtaatgttttttttttgttctcgaacattttgaataaatctgcagacagatggggacgcgtaatttgaatttgtttttcagcgttcttttgtcactggccccttgtttaagtggaaccgcgttgcaatgcggcgagggcatttctctgttttgatttccttctttttctcctttgtgtttcttcaatttgacggtttgcacgctgtgcggtggagcgttttcccttgtgaaataagggccaactgcttcacagtggcacgagggcgctcaagagatccgcgggtcgccagtgactcactttgtgtggcgcagctcgaggaggtgtctggctgctgtgggggcctcgatggttgccacttcgcgagtttgcaacgagcgtgcttctcgcggagggagcaggcgaaatattttgttttttttttttgttttttttgtttttttgtttttgtgtgtgtgtgtaagttttggttcagtctcccttgaactgggggacgttgggcttaatggaccaaactctgattcccctaaaacttcttttgttggttttcttttgtttttgtttttgtgctgctgatttgcacgctttctcactgtcaccgaagcgcggcggcggtgtttgagtgccccctcacgctgctgctgtggaatttgcgttgcttgcggacatttctgttgggtcgcattgctttctacttcgttttttatttttgtggtttggtggaggggagtgtgcagcagggggcgggccgagatgcctgtggagacagcgacgttgcggggactctctctcggcctcgtcattcaacaatccattgcgcagcaggttgccacgaacaccagcaccaatatttgttcgttttcccactattaccggcgcgtctagccgcacgatgccatctgggtgccgaggaggcggttgagcagcggaaaaggcttcctgctatgaagcgactgccattgagagaacttttagctgcgtggatcttcctcaatgcccagccgttggcgcgcagcggaggtgcctgggcattctaggagcagatggcgaaaggtttcctgcgcgtcaactggcgtgtctgtggaggttggctatcctcagtcgggagaccgcctcctggcaccacagaacgggtagcggtagtgtcttggcgaatagtacaacgccacttgttgctgactgggcagtaaagcatgtcagcgggtccgtgtgccatacgggcgcattccatgttccgtgtgttgtccggttgccatggtctgcgtcgcatgctgagccgcaggctcgtcaacatgcactccacaatgtccgtaagaaaactcccggtgcac

SEQ ID NO: 4 - protein, 1162 aa, Trypanosoma cruzi
MVAIADARYETSSENSLIDTVAKYSVDDGETWETQIAIKNSRVSSVSRVVDPTVIVKGNKLYVLVGSYYSSRSYWSSHGDARDWDILLAVGEVTKSTAGGKITASIKWGSPVSLKKFFPAEMEGMHTNQFLGGAGVAIVASNGNLVYPVQVTNKRKQVFSKIFYSEDDGKTWKFGKGRSDFGCSEPVALEWEGKLIINTRVDWKRRLVYESSDMEKPWVEAVGTVSRVWGPSPKSNQPGSQTSFTAVTIEGMRVMLFTHPLNFKGRCVRDRLNLWLTDNQRIYNVGQVSIGDENSAYSSVLYKDDKLYCLHEINTDEVYSLVFARLVGELRIIKSVLRSWKNWTATCPAFAPLLIQPLRRQRVVVVPLSPRLVLLAFCRQRLPKRMGGSYRCVNASTANAERVRNGLKFAGVGGGALWPVSQQGQNQRYRFANHAFTLVASVTIHEAPRAASPLLGASLDSSGGKKLLGLSYDEKHQWQPIYGSTPVTPTGSWETGKRYHLVLTMANKIGSVYIDGELLEGSGQTVVPDGRTPDISHFYVGGYKRSDMPTISHVTVNNVLLYNRRQLNTEEIRTLFLSQDLIGTEAHMDSSSDSSAHSTPSTPADSSAHSTPSTPVDSSAHSTPSTPADSSAHGTPSTPVDSSAHGTPSTPADSSAHGTPSTPVDSSAHSTPSTPVDSSAHSTPSTPVDSSAHGAPSTPADSSAHGTPSTPVDSSAHGTPSTPADSSAHSTPSTPADSSAHSTPSTPADSSAHSTPSTPVDSSAHGTPSTPADSSAHSTPSTPADSSAHGTPSTPVDSSAHSTPSTPVDSSAHGTPSTPVDSSAHSTPSTPVDSSAHGTPSTPVDSSAHSTPSTPADSSAHSTPSTPADSSAHGTPSTPVDSSAHSTPSTPADSSAHSTPSTPVDSSAHSTPSTPADSSAHGTPSTPVDSSAHGTPSTPADSSAHSTPSTPADSSAHSTPSTPADSSAHSTPSTPVDSSAHSTPSTPADSSAHSTPSTPADSSAHSTPSTPADSSAHSTPSTPVDSSAHSTPSTPADSSAHGTPSTPADSSAHSTPSTPVDSSAHSTPSTPADSSAHGTPSTPADSSAHSTPSTPADSSAHGTPSTPADSSAHSTPSTPAGSSANGTVLILPDGAALSTFSGGGLLLCACALLLHVFFMAVF

SEQ ID NO: 5 - protein, 634 aa, Trypanosoma cruzi
LAPGSSRVELFKRKNSTVPFEDKAGKVTERVVHSFRLPALVNVDGVMVAIADARYDTSNDNSLIDTVAKYSVDDGETWETQIAIKNSRVSSVSRVVDPTVIVKGNKLYVLVGSYYSSRSYWSSHGDARDWDILLAVGEVTKSTAGGKITASIKWGSPVSLKKFFPAEMEGMHTNQFLGGAGVAIVASNGNLVYPVQVTNKKKQVFSKIFYSEDDGKTWKFGKGRSDFGCSEPVALEWEGKLIINTRVDWKRRLVYESSDMEKPWVEAVGTVSRVWGPSPKSNQPGSQSSFTAVTIEGMRVMLFTHPLNFKGRWLRDRLNLWLTDNQRIYNVGQVSIGDENSAYSSVLYKDDKLYCLHEINTDEVYSLVFARLVGELRIIKSVLRSWKNWDSHLSSICTPADPAASSSESGCGPAVTTVGLVGFLSGNASQNVWEDAYRCVNASTANAERVRNGLKFAGVGGGALWPVSQQGQNQRYRFANHAFTLVASVTIHEAPRAASPLLGASLDSSGGKKLLGLSYDEKHQWQPIYGSTPVTPTGSWETGKRYHLVLTMANKIGSVYIDGELLEGSGQTVVPDGRTPDISHFYVGGYKRSDMPTISHVTVNNVLLYNRQLNTEEIRTLFLSQDLIGTEAHM

SEQ ID NO: 6 - protein, 7 aa, Trypanosoma cruzi
NSRVSSV

SEQ ID NO: 7 - protein, 7 aa, Trypanosoma cruzi
KRKNSTV

SEQ ID NO: 8 - protein, 7 aa, Trypanosoma cruzi
RVMLFTH

SEQ ID NO: 9 - protein, 7 aa, Trypanosoma cruzi
RSDMPTI

SEQ ID NO: 10 - protein, 7 aa, Trypanosoma cruzi
RSYWSSH

SEQ ID NO: 11 - DNA, 30 bp, Artificial Sequence
  Description of Artificial Sequence Synthetic primer
ccgctcgagatgggtttggcacccggatcg

SEQ ID NO: 12 - DNA, 27 bp, Artificial Sequence
  Description of Artificial Sequence Synthetic primer
tccccgcggtcagaaaactgccataaa

SEQ ID NO: 13 - protein, 12 aa, Trypanosoma cruzi
DSSANGTPSTPA

SEQ ID NO: 14 - protein, 6 aa, Artificial Sequence
  Description of Artificial Sequence Synthetic peptide
RXRXXX

SEQ ID NO: 15 - protein, 7 aa, Artificial Sequence
  Description of Artificial Sequence Synthetic peptide
RXRXXXX

SEQ ID NO: 16 - protein, 4 aa, Artificial Sequence
  Description of Artificial Sequence Synthetic peptide
PXXP

SEQ ID NO: 17 - protein, 4 aa, Trypanosoma cruzi
YWTD

(19) United States
(12) Patent Application Publication
(10) Pub. No.: US 2012/0245221 A1
(43) Pub. Date: Sep. 27, 2012

(54) ANTI-APOPTOTIC AGENTS AND USES THEREOF

(75) Inventors: Marina V. Chuenkova, Boston, MA (US); Mercio A. Perrin, Chestnut Hill, MA (US)

(73) Assignee: TUFTS University, Medford, MA (US)

(21) Appl. No.: 13/505,316

(22) PCT Filed: Nov. 5, 2010
(86) PCT No.: PCT/US10/55700

§ 371 Date: May 30, 2012

Related U.S. Application Data
(60) Provisional application No. 61/258,961, filed on Nov. 6, 2009.

(51) Int. Cl. A61K 48/00; C12N 15/861; C12N 15/86; C12N 15/85

(52) U.S. Cl. 514/44 R; 435/455

(57) ABSTRACT

The invention generally relates to the use of parasite-derived neurotrophic factor (PDNF), or a fragment of PDNF, to reduce cell apoptosis. The PDNF or PDNF fragment is provided in the cytoplasm of a cell so that it can bind to Akt kinase and induce Akt kinase activation.

### CROSS REFERENCE TO RELATED APPLICATIONS

This application claims the benefit of U.S. Provisional Application No. 61/258,961, filed Nov. 6, 2009, which is incorporated by reference in its entirety.

### GOVERNMENT SUPPORT

The work described herein was funded, in whole or in part, by grant numbers 5R01NSO40574-09 and 5R01NSO42960-08 from the National Institutes of Health. The United States government may have certain rights in the invention.

### BACKGROUND OF THE INVENTION

Apoptosis, or programmed cell death, is required for normal development, tissue homeostasis and the elimination of damaged cells. However, an increase or decrease in apoptosis may contribute to the pathology of a wide range of disorders and diseases, including Alzheimer's disease (AD), Parkinson's disease (PD), Huntington disease (HD), amyotrophic lateral sclerosis (ALS), ischemia and stroke, cardiovascular diseases, inflammatory diseases, spinal cord trauma, and head injury.

Neurotrophic factors (NTFs) are a group of proteins that can regulate the survival, development, differentiaiton and many of the functions of neural cells. Several neurotrophic factors have been described including members of the NGF-family of neurotrophins, such as nerve growth factor (NGF), brain-derived neurotrophic factor (BNGF), neurotrophin-3 (NT-3) and neurotrophin-4 (NT-4), and members of the IL-6 family, including interleukin-6 (IL-6), interleukin-11 (IL-11), leukemia inhibitory factor (LIF), cilliary neurotrophic factor (CNTF) and oncostatin-M (OSM). The term “neurotrophin” is sometime used to refer four structurally related factors: NGF, BDNF, NT-3, and NT-4/5.

The discovery that NTFs support neuronal survival and function in the adult central nervous system (CNS) has generated broad interest in the use of these factors to treat neurodegenerative diseases. Numerous in vitro studies and in vivo studies in animal models of neuronal degeneration have provided proof-of-concept and preclinical data that have led to several clinical trials either using peripheral or intracerebroventricular (i.c.v.) protein administration, or using more sophisticated means of NTF delivery such as gene therapy. Indeed, it has become increasingly clear that the successful implementation of NTF therapy may require a targeted, localized delivery of NTFs to avoid unwanted adverse effects resulting from widespread receptor activation. These insights have led to the first promising clinical trials of NTFs in AD and PD. See, e.g., Blesch, Neurotrophic Factors in Neurodegeneration, Brain Pathology, 16:295-303 (2006).

NGF was the first NTF that was discovered in the search for neuron survival-promoting factors in the nervous system. A small clinical trial using i.c.v. infusions of NGF for treating AD was discontinued because of the development of a pain syndrome in some patients. Subsequently, a means of localized, intraparenchymal NGF delivery was developed using cells genetically modified to express NGF, and a phase I study of ex vivo NGF gene therapy was initiated. Cognitive testing indicated an improvement in the rate of cognitive decline, in particular after longer time periods post surgery. See, e.g., Blesch, supra.

Improvements in gene therapy and vector design also made it possible to inject replication-incompetent viral vectors (such as adeno-associated virus (AAV) or lentivirus) directly in vivo. Studies using direct in vivo NGF gene transfer in animal models showed localized production of NGF and confirmed its neuroprotective effects on basal forebrain cholinergic neurons. See, e.g., Blesch, supra.

Glial cell-line derived neurotrophic factor (GDNF) and other members of the same family were found to have potent effects on dopaminergic neuronal survival. A phase I trial injecting GDNF i.c.v. for treating PD turned out to be ineffective, and resulted in severe adverse effects. The lack of efficacy resulted from insufficient diffusion of GDNF from the lateral ventricle to the actual target area, the striatum. See, e.g., Blesch, supra.

U.S. Application Publication No. 20020187951 discloses methods for treating or preventing neurodegenerative diseases by administering a lentiviral vector that expresses GDNF.

In excitotoxic lesion models of Huntington's Disease (HD), several NTFs have been reported to be neuroprotective to a variable degree, including NGF, BDNF, NT-3, NT-4/5, GDNF, transforming growth factor-β, and the neuropoetic cytokine CNTF. The mechanism of some of the neuroprotective effects observed is not fully established and might be indirect. Studies have also been conducted to evaluat NTF gene transfer using AAV or lentivirus as a means to provide long-term, localized NTF support in animal models of HD. Overexpression of BDNF, GDNF and CNTF using AAV, adenovirus or lentivirus were found to be protective after excitotoxic lesions. See, e.g., Blesch, supra.

BDNF, CNTF, insulin-like growth factor-1 (IGF-1) and GDNF have also been evaluated in animal models of motor neuron disease or ALS by direct protein delivery or via gene therapy vectors. Expression of vascular endothelial growth factor (VEGF) in motor neurons via retrograde transport of a VEGF-coding lentivirus from muscle and expression of IGF-1 in motor neurons after AAV-2 injection into muscle improved animal survival and delayed motor neuron death in transgenic mice overexpressing mutant superoxide dismutase-1 (SOD-1). GDNF expressed in muscle after AAV gene transfer was also shown to be retrogradely transported to motor neurons, to delay motor neuron degeneration and to prolong the lifespan of SOD-1 expressing mice. See, e.g., Blesch, supra.

Changes in NTF signaling are believed to contribute to neuronal degeneration in some CNS disorders. Expression of the NTFs and their respective receptors can be altered in several different diseases or injury states that impact upon the functions in the central and peripheral nervous systems. The intracellular signals used by neurotrophins are triggered by ligand binding to the cell surface Trk and p75^{NTR} receptors. In general, activation of Trk receptors support survival, growth and synaptic strengthening, while activation of p75^{NTR} induces apoptosis, attenuates growth and weakens synaptic signaling. See, e.g., Twiss, et al., Pathophysiological Mechanisms for Actions of the Neurotrophins, Brain Pathology, 16:320-332 (2006); Huang & Reichardt, Neurotrophins: Roles in Neuronal Development and Function; Annu Rev Neurosci. 2001; 24: 677-736.

Many disease, including Alzheimer's disease, Parkinson's disease, Huntington disease, Amyotrophic Lateral Sclerosis, ischemia and stroke, spinal cord trauma and head injury, cardiovascular diseases and inflammatory diseases, are associated with apoptosis. Despite the growing amount of information regarding the pathological and molecular mechanisms that lead to the apoptotic cell death, signal transduction pathways that contribute to the cell death have not been fully elucidated.

A need exists for reducing or inhibiting cell apoptosis, in particular for the purpose of treating diseases or disorders that are associated with cell apoptosis.

### SUMMARY OF THE INVENTION

The invention generally relates to methods of reducing cell apoptosis using parasite-derived neurotrophic factor (PDNF), or a fragment of PDNF. In particular, the invention provides methods of reducing cell apoptosis by delivering to a cell a nucleic acid molecule that encodes PDNF (or a fragment of PDNF), so that the PDNF or PDNF fragment can be provided intracellularly. The methods may be used to treat diseases that are associated with cell apoptosis, in particular neurodegenerative diseases such as Alzheimer's disease, Parkinson's disease, or Huntington disease.

In one aspect, the invention provides a method of reducing cell apoptosis, comprising: delivering to a cell a nucleic acid molecule comprising a nucleotide sequence that encodes parasite-derived neurotrophic factor (PDNF), or a fragment of PDNF, wherein the PDNF or PDNF fragment binds to Akt kinase. Preferably, the PDNF or PDNF fragment does not comprise a secretory signal peptide sequence.

In certain embodiments, the PDNF or PDNF fragment comprises an Akt kinase phosphorylation site. In certain embodiments, the phosphorylated PDNF or PDNF fragment induces activation of Akt kinase (for example, an increase in the kinase activity of Akt kinase, an increase in the expression level of a gene encoding Akt kinase, or both).

In certain embodiments, the PDNF comprises an amino acid sequence that is selected from the group consisting of: (a) the amino acid sequence set forth in SEQ ID NO: 5; (b) the amino acid sequence set forth in residues 1 to 588 of SEQ ID NO: 4; and (c) an amino acid sequence that is at least 85%, at least 90%, or at least 95% identical to (a) or (b).

In certain embodiments, the phosphorylation site of the PDNF or PDNF fragment is a serine or threonine residue that corresponds to positions S91, T17, T304, T597, or 5123 of SEQ ID NO:5 or a fragment of SEQ ID NO:5. In certain embodiments, the phosphorylation site of the PDNF or PDNF fragment is 591, T17, T304, T597, or 5123 of SEQ ID NO:5 or a fragment of SEQ ID NO:5.

In certain embodiments, the nucleic acid molecule of the invention comprises a nucleotide sequence that is selected from the group consisting of: (a) nucleotides 234 to 2123 of the nucleotide sequence set forth in SEQ ID NO: 1; (b) nucleotides 484 to 2248 of the nucleotide sequence set forth in SEQ ID NO: 3; and (c) a nucleotide sequence that is at least 85%, at least 90%, or at least 95% identical to (a) or (b).

In certain embodiments, the nucleic acid molecule is a vector derived from an adenovirus, an adeno-associated virus, a lentivirus, or an alphavirus. In certain embodiments, the nucleic acid molecule is a replication-deficient viral vector.

In certain embodiments, the nucleic acid molecule is a vector comprising a nucleotide sequence that encodes the PDNF or PDNF fragment that is operably linked to an expression control sequence (such as a promoter, an enhancer, a ribosome entry site, or a polyadenylation sequence) that promotes the expression of the PDNF or PDNF fragment in a mammalian cell.

In certain embodiments, the nucleic acid molecule is administered to a mammalian subject (such as a human) in need of reducing cell apoptosis. In certain embodiments, the mammalian subject is in need of reducing apoptosis of neurons or glial cells (such as Schwann cells). In certain embodiments, the subject is suffering from or susceptible to a neurodegenerative disease.

In another aspect, the invention provides a method of reducing the effect of an apoptotic-inducing agent on a mammalian subject (such as a human), comprising: administering to the subject in need thereof a nucleic acid molecule comprising a nucleotide sequence that encodes PDNF, or a fragment of PDNF, wherein the PDNF or PDNF fragment binds to Akt kinase. Preferably, the PDNF or PDNF fragment does not comprise a secretory signal peptide sequence. In certain embodiments, the PDNF or PDNF fragment comprises an Akt kinase phosphorylation site. In certain embodiments, the subject is in need of reducing the effect of an apoptotic-inducing agent on neurons or glial cells.

Examples of apoptotic-inducing agents include inducers such as deprivation of a growth factor, pro-inflammatory cytokines, free radicals, oxidative stress, Fas ligand, anti-Fas antibody, staurosporine, Tumor Necrosis Factor, ultraviolet and gamma-irradiation.

In certain embodiments, the apoptotic-inducing agent is a cytokine, such as a pro-inflammatory cytokine (e.g., TGF-β or TNF-α). In certain embodiments, the apoptotic-inducing agent can cause oxidative stress. In certain embodiments, the apoptotic-inducing agent can produce H₂O₂ or free radicals.

In certain embodiments, the phosphorylated PDNF or PDNF fragment induces suppression of pro-apoptotic activities (for example, a decrease in the activity of a pro-apoptotic protein, a decrease in the expression level of a gene encoding a pro-apoptotic protein, or both). Examples of pro-apoptotic proteins include, e.g., Caspase-9, FOXO, or BAX.

In another aspect, the invention provides a method of activating Akt kinase in a cell, comprising: delivering to the cell a nucleic acid molecule comprising a nucleotide sequence that encodes parasite-derived neurotrophic factor (PDNF), or a fragment of PDNF, wherein the PDNF or PDNF fragment binds to Akt kinase. Preferably, the PDNF or PDNF fragment does not comprise a secretory signal peptide sequence.

In another aspect, the invention provides a method of treating a condition in a mammalian subject wherein the condition is alleviated by increased activity of Akt kinase, comprising: delivering to the subject a nucleic acid molecule comprising a nucleotide sequence that encodes PDNF, or a fragment of PDNF, wherein the PDNF or PDNF fragment binds to Akt kinase. Preferably, the PDNF or PDNF fragment does not comprise a secretory signal peptide sequence.

The invention also relates to the PDNF or PDNF fragment as described herein for use in therapy (e.g., for treating a neurodegenerative disease), and to the use of the PDNF or PDNF fragment for the manufacture of a medicament for reducing cell apoptosis (e.g., for treating a neurodegenerative disease).

### BRIEF DESCRIPTION OF THE DRAWINGS

FIG. 1 shows that PDNF interacts with Akt and with an antibody against Akt-phosphorylated substrates in T. cruzi-infected Schwann cells. FIG. 1A shows the motifs within PDNF that are targets for phosphorylation by Akt. The N-terminal region of PDNF (solid line) shows the phosphorylation sites and the C-terminal proline-rich region of tandem 12 amino acid residue repeats (hatched line) (19, 20). In a 3-dimensional (3D) structure of PDNF (amino acid residues 1 to 124), Thr¹⁷ and Ser⁹¹ are located on the surface of PDNF, and the Thr¹⁷- and Ser⁹¹-containing motifs each has a β-turn that is located on the surface of PDNF (59). FIG. 1B shows that PDNF coimmunoprecipitates with Akt. Lysates of uninfected (Sc) or T. cruzi-infected (Sc-Inf) Schwann cells (4 days PI) were immunoprecipitated (IP) with the PDNF-specific mAb TCN-2, followed by Western blotting analysis (WB) with TCN-2 or an antibody against Akt (αAkt). FIG. 1C shows that PDNF coimmunoprecipitates with pAkt and Akt-phosphorylated substrates. Lysates of Sc cells and Sc-Inf cells immunoprecipitated with TCN-2 were incubated with an antibody against Akt-phosphorylated substrates (left, αP-Akt sbstr)) or with TCN-2 (right). The same lysates immunoprecipitated with an antibody against Akt-phosphorylated substrates were incubated with TCN-2 (middle).

FIG. 2 shows that Akt phosphorylates PDNF in T. cruzi-infected cells and in vitro. FIG. 2A shows the Akt-dependent phosphorylation of PDNF in infected cells. Uninfected (Sc) or T. cruzi-infected (Sc-Inf) Schwann cells were treated with vehicle (O), with the PI3K inhibitor LY294002, or with the Akt inhibitor Akti VIII for 24 hours prior to harvesting. Cells were lysed and lysates were incubated with antibodies against pAkt (Ser⁴⁷³) or, after immunoprecipitation with an antibody against PDNF (TCN-2), with antibodies specific for Akt-phosphorylated substrates (αP-Akt sbstr). FIG. 2B shows that Akt phosphorylates PDNF in vitro. Bacterially-expressed PDNF (bPDNF) was used as a substrate for immuno-purified Akt in kinase reactions with (+) or without (−) ATP. Phosphorylation of PDNF was analyzed with an antibody specific for Akt-phosphorylated substrates. Cs, Coomassie blue-stained bPDNF. Identical results were obtained in at least three experiments.

FIG. 3 shows the developmental regulation of the activation of Akt in T. cruzi-infected Schwann cells. In FIG. 3A, lysates of Schwann cells, uninfected (Sc) or infected with T. cruzi (Sc-Inf), were incubated with antibodies specific for pAkt (Ser⁴⁷³), total Akt, or Akt-phosphorylated substrates (P-Akt-sbstr). Inset shows the ratio of pAkt to total Akt in the blot. FIG. 3B shows the activation of Akt increases with the progress of the infection. Uninfected Schwann cells (Sc) or Schwann cells infected with T. cruzi (Sc-Inf) for 3, 4, or 5 days, were lysed and analyzed by Western blotting with antibodies specific for PDNF (TCN-2), pAkt (Ser⁴⁷³) (αP-Akt), and total Akt (αAkt). The bar graph shows the ratio of pAkt to total Akt for this blot. At 5 days, Schwann cells were filled with a mixture of amastigotes and mobile trypomastigotes. FIG. 3C shows that the trans-sialidase activity of PDNF increases with the progression of infection. Specific trans-sialidase activity was determined in lysates of infected Schwann cells (same samples as in Panel B).

FIG. 4 shows that PDNF in the cytosol of Schwann cells activates Akt. In FIG. 4A, lysates of Schwann cells transfected with empty vector (Sc-Red) or with a plasmid encoding PDNF (Sc-PDNF) were analyzed by Western blotting with antibodies against PDNF, pAkt (Ser⁴⁷³), pAkt (Thr³⁰⁸), and Akt. Right, the ratio of pAkt to Akt in the lysates of Sc-Red and Sc-PDNF cells was determined by scanning densitometry of the blot. FIG. 4B shows the specific trans-sialidase activity of infected and transfected Schwann cells. Specific enzyme activity in lysates of Schwann cells infected with T. cruzi (Sc-Inf) and in lysates (L) and culture supernatants (S) of Schwann cells transfected with plasmid encoding PDNF (Sc-PDNF) or with empty vector (Sc-Red). FIG. 4C shows that Akt phosphorylates PDNF in transfected cells. Schwann cells transfected with empty vector (Sc-Red) or with a plasmid encoding PDNF (Sc-PDNF) were treated with the Akt inhibitor Akti VIII for 24 hours prior to harvesting. Cell lysates were immunoprecipitated with the PDNF-specific antibody TCN-2 and samples were then incubated with antibodies against Akt-phosphorylated substrates (αP-Akt sbstr). All the experiments were repeated at least three times with similar results.

FIG. 5 shows that expression of PDNF in the cytosol of Schwann cells augments transcription of the gene encoding Akt and reduces the transcription of genes encoding pro-apoptotic factors. FIG. 5A shows the results from microarray analyses, which are expressed as the ratio of signals from mRNA isolated from Sc-PDNF cells to those of Sc-Red cells. This experiment was repeated twice with similar results. PKA, protein kinase A; PKCβ, protein kinase Cβ; CMK2, cytidine monokinase 2; Tousled, tousled kinase. FIG. 5B shows the result of qPCR analysis of the expression of the genes encoding the three isoforms of Akt in Sc-Red cells and Sc-PDNF cells. Results represent the ratio of Akt mRNA relative to that of hypoxanthine-guanine phosphoribosyltransferase (HPRT). Results are presented as the mean±the standard deviation (SD) of 4 independent experiments. **, p<0.05.

FIG. 6 shows that Schwann cells transfected with a plasmid encoding PDNF or infected with T. cruzi exhibit Akt-dependent resistance to oxidative stress. In FIG. 6a, infected cells were pretreated with the Akt inhibitor Akti VIII (10 μM) before treatment with 100 μM H₂O₂ for 24 hours. Cell survival was assessed with an MTT-based assay. In FIG. 6b, Schwann cells transfected with empty vector (Sc-Red) or with a plasmid encoding PDNF (Sc-PDNF) were treated and analyzed for cell survival as described for panel B. ***, p<0.001 of three experiments.

FIG. 7 shows that Schwann cells transfected with a plasmid encoding PDNF are protected from apoptosis induced by TNF-α and TGF-β1. Sc-Red and Sc-PDNF cells were left untreated or were pretreated with the Akt inhibitor Akti VIII (10 μM), cultured for 4 days in DMEM, 1% BSA containing TNF-α (20 ng/ml) and TGF-β1 (40 ng/ml), and analyzed for apoptosis by the TUNEL assay (as described for FIG. 6). Apoptosis was quantified as % of the total number of cells that were TUNEL-positive as revealed by DAPI staining. ***, p<0.001 of three experiments.

FIG. 8 shows the amino acid sequence of Akt phosphorylation motifs. Phosphorylation sites (marked by S or T underlined) and sequences are indicated. Percentile represent medium stringency for Akt phosphorylation sites.

FIG. 9 provides the nucleotide and amino acid sequences of exemplary PDNFs. FIG. 9A illustrates the nucleotide sequence of the PDNF gene, clone 19Y (SEQ ID NO:1) deposited in GenBank under accession number AJ002174, having an open-reading frame beginning at position 370. FIG. 9B illustrates the amino acid sequence of the PDNF (SEQ ID NO:2) encoded by clone 19Y deposited in GenBank under accession number AJ002174. FIG. 9C illustrates the nucleotide sequence of the PDNF gene, clone 7F (SEQ ID NO:3) deposited in GenBank under accession number M61732, having an open-reading frame beginning at position 484. FIG. 9D illustrates the amino acid sequence of the PDNF (SEQ ID NO:4) encoded by clone 7F deposited in GenBank under accession number M61732. The PDNF comprises a catalytic domain (amino acid residues 33-666 of SEQ ID NO:4), and a tandem repeat domain (amino acid residues 667-1162 or SEQ ID NO:4). FIG. 9E illustrates the amino acid sequence of a fragment of PDNF encoded by clone 19Y in which the secretory signal peptide sequence has been deleted (SEQ ID NO:5, which correspond to amino acid residues 333-666 of SEQ ID NO: 2).

### DETAILED DESCRIPTION OF THE INVENTION

### 1. Overview

The invention generally relates to methods of reducing cell apoptosis using parasite-derived neurotrophic factor (PDNF), or a fragment of PDNF. In particular, the invention provides methods of reducing cell apoptosis by delivering to a cell a nucleic acid molecule that encodes PDNF (or a fragment of PDNF), so that the PDNF (or PDNF fragment) can be provided intracellularly. The methods may be used to treat diseases that are associated with cell apoptosis, in particular neurodegenerative diseases such as Alzheimer's disease, Parkinson's disease, or Huntington disease.

The invention is based in part on the discovery that in the cytosol, PDNF is a substrate and an activator of the serine-threonine kinase Akt, a prototypic anti-apoptotic enzyme. Like many other growth factors, PDNF had been shown to activate signal transduction pathways extracellularly, by binding to cell surface receptors such as the TrkA receptor. However, the intracellular anti-apoptotic activities of PDNF have not been previously described or investigated.

As described and exemplified herein, the inventors studied the intracellular activities of PDNF, and found that intracellular PDNF induced (1) increased expression of the gene that encodes Aid; and (2) decreased expression of genes that encode pro-apoptotic factors (such as Caspase-9, BAX, and FOXO). Consequently, in cell culture models in which apoptosis was induced by oxidative stress and cytokines (tumor necrosis factor-α and transforming growth factor-β), intracellular PDNF was shown to elicit sustained protection from apoptosis.

Therefore, in one aspect, the invention relates to methods of reducing cell apoptosis using PDNF (or a fragment of PDNF), wherein the PDNF is provided intracellularly, and therefore, can bind to Akt kinase. In preferred embodiments, a nucleic acid molecule encoding the PDNF (or PDNF fragment) is delivered to the cell, so that the PDNF (or PDNF fragment) is synthesized in the cytoplasm. Preferably, the PDNF or PDNF fragment does not comprise a secretory signal peptide sequence so that the PDNF or PDNF fragment remains in the cytoplasm.

In the cytoplasm, the phosphorylation of the PDNF or PDNF fragment can induce an increase in Akt kinase activity, an increase in the expression level of Akt kinase gene, a decrease in the activity of a pro-apoptotic protein (such as Caspase-9, FOXO, or BAX), a decrease in the expression level of pro-apoptotic genes (such as genes encoding Caspase-9, FOXO, or BAX), or a combination thereof, thereby reducing apoptosis of the host cell.

The PDNF or PDNF fragment can be administered to a suitable mammalian subject (such as a human) to reduce apoptosis. In particular, many neurological diseases are caused by the death of neurons and/or glial cells. Reducing apoptosis can prevent or delay the onset or progression of the diseases, mitigate the severity of the diseases, or protect the cells from further damages.

In another aspect, the invention also relates to methods of reducing the effect of an apoptotic-inducing agent on a mammalian subject using PDNF (or a fragment of PDNF), wherein the PDNF (or PDNF fragment) binds to Aid kinase. In preferred embodiments, a nucleic acid molecule encoding the PDNF (or PDNF fragment) is delivered to a cell of the subject, so that the PDNF (or PDNF fragment) is synthesized in the cytoplasm. Preferably, the PDNF or PDNF fragment does not comprises a secretory signal peptide sequence so that the PDNF or PDNF fragment remains in the cytoplasm.

For example, pro-inflammatory cytokines and free radicals can cause apoptosis. The increased incidence of neurodegenerative diseases may be attributed to a pro-oxidative environment caused by smoking, alcohol abuse, UVA and UVB radiations, air pollution as well as inappropriate nutrition. The harmful effect of oxidative stress can be mitigated by the administration of PDNF or PDNF fragment that functions intracellularly to activate Akt kinase.

Another aspect of the invention relates to methods of activating Akt kinase in a cell using PDNF (or a fragment of PDNF), wherein the PDNF or PDNF fragment binds to Akt kinase.

Another aspect of the invention relates to methods of treating a condition in a subject wherein the condition is alleviated by an increased activity of Akt kinase using PDNF (or a fragment of PDNF), wherein the PDNF or PDNF fragment binds to Akt kinase.

### 2. Definitions

As used herein, the singular forms “a,” “an” and “the” include plural references unless the content clearly dictates otherwise.

The term “about”, as used here, refers to +/−10% of a value.

As used herein, the term “vector” refers to a genetic element, such as a plasmid, phage, transposon, cosmid, chromosome, virus, virion, etc., that is capable of carrying and/or expressing exogenous nucleic acid sequences, and optionally is capable of replication when associated with the proper control elements. Thus, the term includes cloning and expression vehicles, as well as viral vectors.

The term “non-viral vector” refers to an autonomously replicating, extrachromosomal nucleic acid molecule that is distinct from the genome of the host cell, and is not assembled into a viral particle or capsid by a host cell.

The term “viral vector” means a recombinant virus that has had some or all of the genes in the native viral genome removed. A viral vector is capable of carrying and expressing exogenous nucleic acid sequences. A viral vector may be replication-deficient, or it may be capable of replication when associated with the proper control elements. The genome of a viral vector typically contains a nucleic acid molecule encoding PDNF (or a fragment of PDNF), so that the viral vector transfers the nucleic acid molecule encoding the PDNF or PDNF fragment to a desired host cell. Representative viral vectors include those that can infect mammalian, and especially human, cells, and are derived from viruses such as retroviruses, adenoviruses, herpes viruses, avipox viruses etc. A viral vector may be a DNA vector or an RNA vector.

The term “replication deficient” or “replication defective” refers to a viral genome that does not comprise all the genetic information necessary for replication and formation of a genome-containing capsid in a replication competent cell under physiologic (e.g., in vivo) conditions.

An amino acid residue of a query sequence “corresponds to” a designated position of a reference sequence (e.g., S91, T17, T304, T597, or S123 of SEQ ID NO: 5) when, by aligning the query amino acid sequence with the reference sequence, the position of the residue matches the designated position. Such alignments can be done by hand or by using well-known sequence alignment programs such as ClustalW2, or “BLAST 2 Sequences” using default parameters.

As used herein, the term “operably linked” refers to a first polynucleotide molecule, such as a promoter, connected with a second polynucleotide molecule, such as a gene of interest, where the polynucleotide molecules are so arranged that the first polynucleotide molecule affects the function of the second polynucleotide molecule. For example, a promoter is operably linked to a gene of interest if the promoter modulates transcription of the gene of interest in a cell.

### 3. Use of Cytoplasmic PDNF to Reduce Cell Apoptosis

The invention generally relates to methods of reducing cell apoptosis using parasite-derived neurotrophic factor (PDNF), or a fragment of PDNF. In one aspect, the invention provides methods of reducing cell apoptosis by delivering to a cell a nucleic acid molecule that encodes PDNF (or a fragment of PDNF), wherein the PDNF or PDNF fragment binds to Akt kinase.

A. Parasite-Derived Neurotrophic Factor (PDNF)

The human parasite Trypanosoma cruzi, the agent of Chagas' disease, expresses a membrane-bound neuraminidase, also known as trans-sialidase (TS), or parasite-derived neurotrophic factor (PDNF) because it binds and activates nerve growth factor (NGF) receptor TrkA in neuronal cells.

PDNF is an enzyme expressed on the T. cruzi's surface and catalyzes the transfer of sialic acid from host glycoconjugates to glycoprotein molecules on the surface of the parasite. See, Schenkman et al., Exp. Parasitol., 72:76 86 (1991). The enzyme is present both in the epimastigote form (i.e., in the invertebrate vector) and in the trypomastigote form (i.e., infectious form that circulates in the blood of the vertebrate host). See, Agusti et al., Glycobiology, 7(6):731 5, (1997).

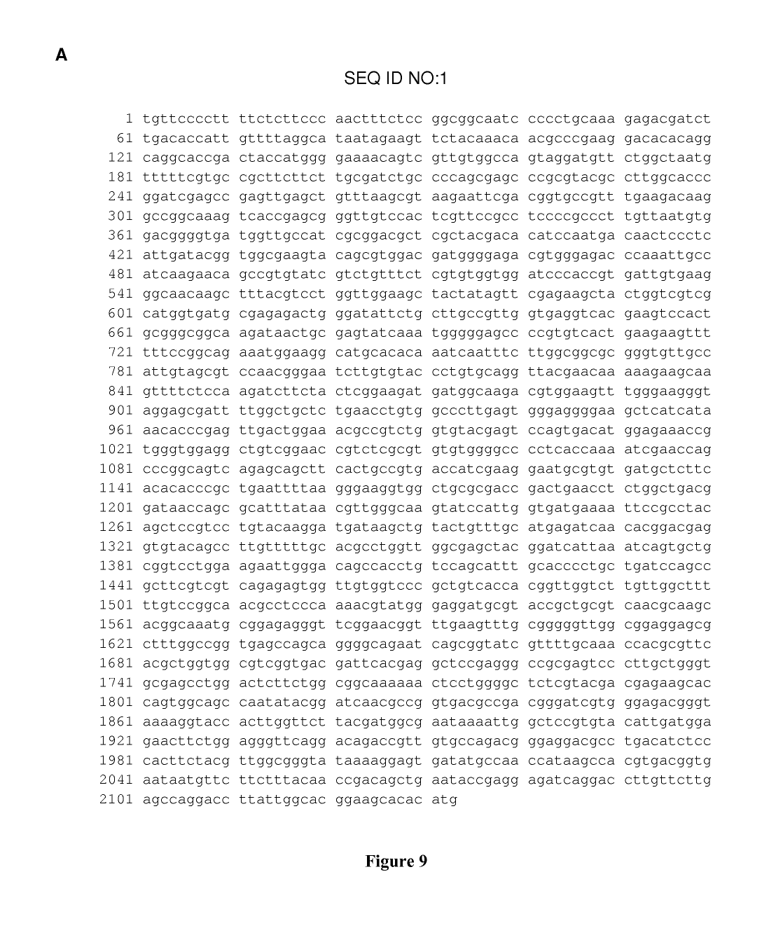

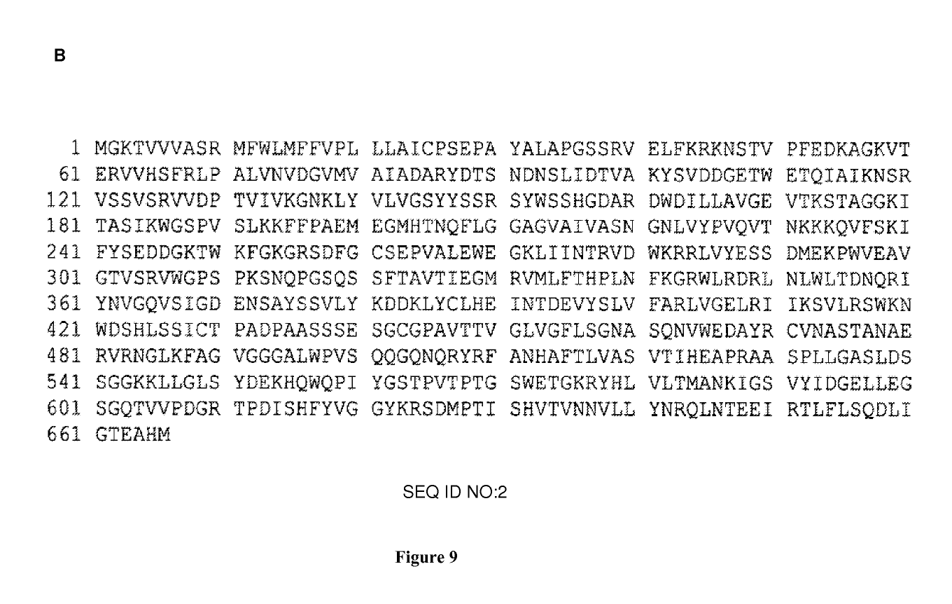

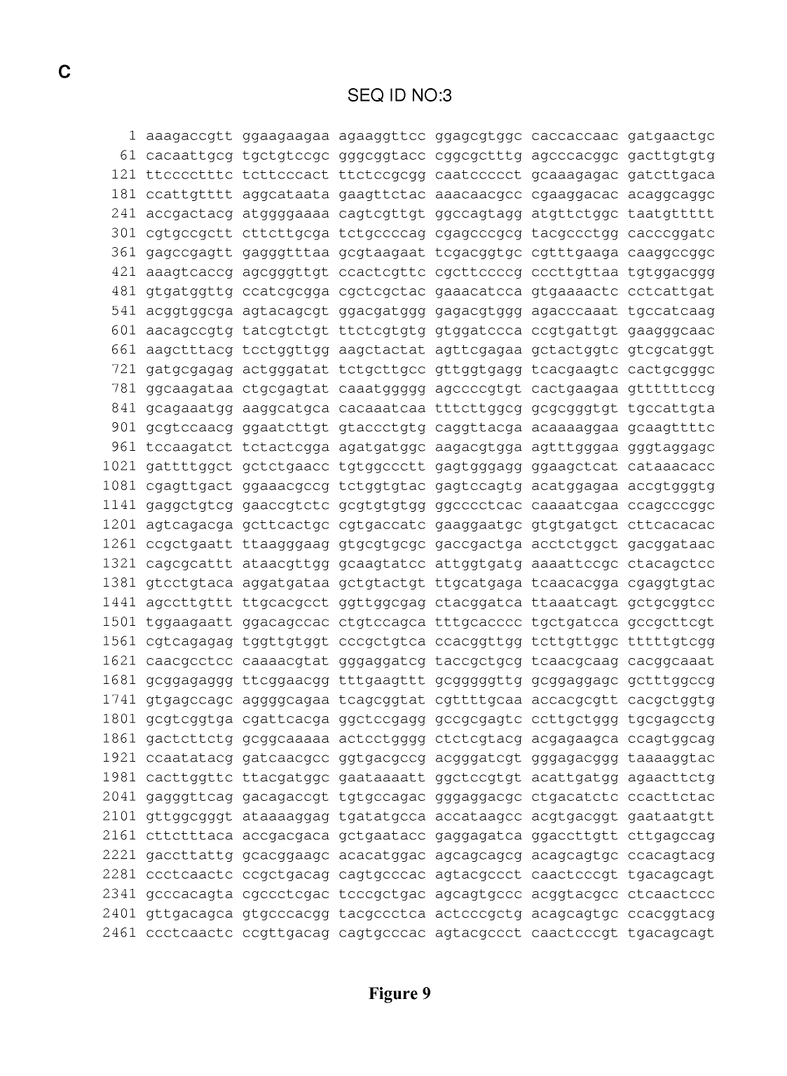

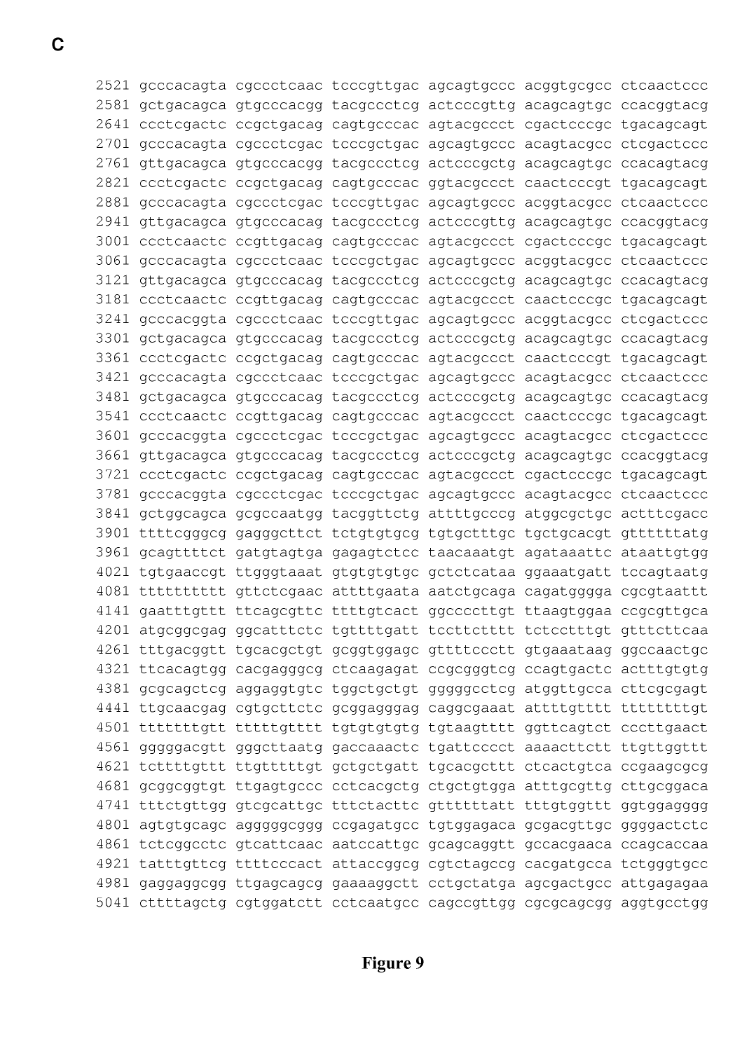

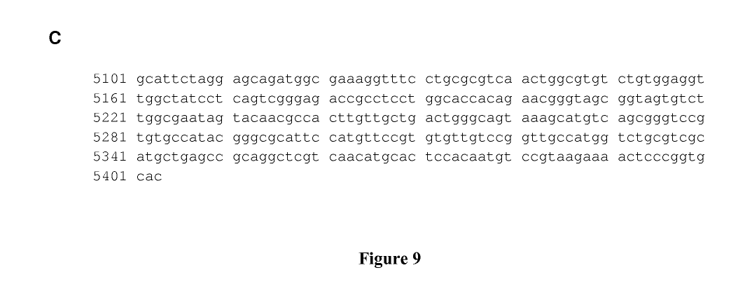

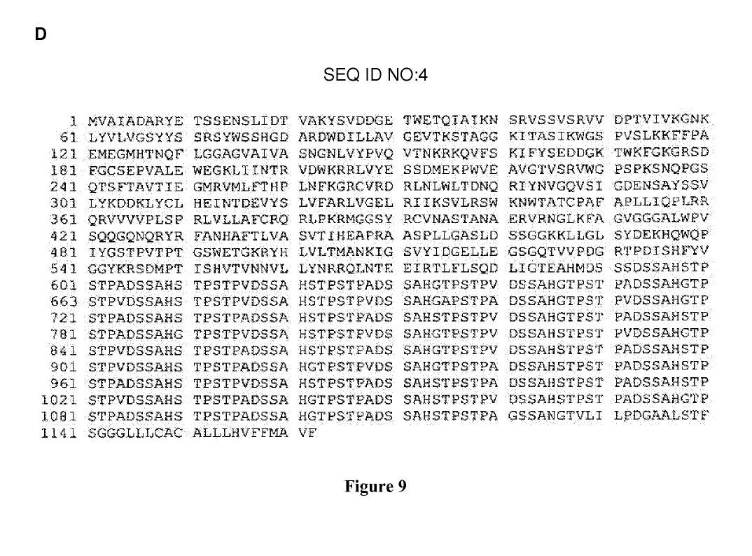

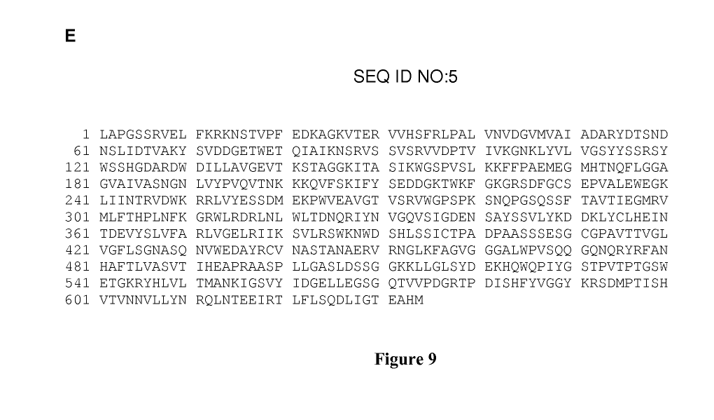

FIG. 9 shows the nucleotide sequences and amino acid sequences of two naturally-occurring PDNFs. FIG. 9A illustrates the nucleotide sequence of the T. cruzi PDNF gene, clone 19Y (SEQ ID NO: 1), deposited in GenBank under accession number AJ002174, having an open-reading frame beginning at position 370. FIG. 9B illustrates the amino acid sequence of the T. cruzi PDNF (SEQ ID NO: 2) encoded by clone 19Y deposited in GenBank under accession number AJ002174. FIG. 9C illustrates the nucleotide sequence of the T. cruzi PDNF gene, clone 7F (SEQ ID NO: 3) deposited in GenBank under accession number M61732, having an open-reading frame beginning at position 484. FIG. 9D illustrates the amino acid sequence of the T. cruzi PDNF (SEQ ID NO: 4) encoded by clone 7F deposited in GenBank under accession number M61732. The entire teachings of the information deposited in GenBank under accession numbers AJ002174 and M61732 are incorporated herein by reference.

The naturally-occurring, full-length PDNF from T. cruzi trypomastigotes has 4 distinct amino acid regions: (1) a N-terminal region with approximately 380 amino acids, which shares about 30% sequence identity to bacterial sialidases; (2) a region with approximately 150 residues that does not show any similarity with any known sequence; (3) a region with homology to type III fibronectin (FnIII); and (4) a C-terminal region containing 12 repeated amino acids, which is the immuno-dominant portion and which is required for enzyme oligomerization. The N-terminal and the FnIII regions are important for trans-sialidase activity.

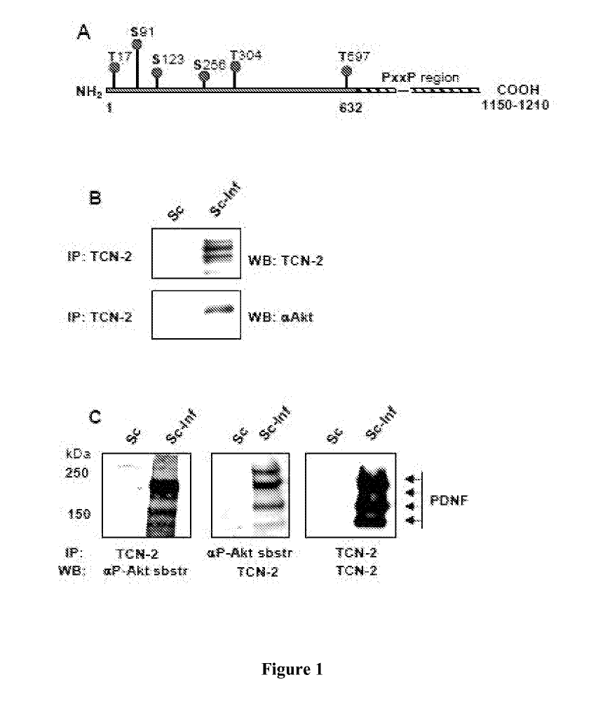

The catalytic portion of a native trans-sialidase has two kinds of enzymatic activities: (1) neuraminidase activity, which releases sialic acid from the complex carbohydrates; and (2) sialil-transferase activity, which catalyzes the transfer of sialic acid from glyconjugate donors to terminal β-D galactose containing acceptors. See, Scudder et al., J. Biol. Chem., 268(13):9886 91 (1993). As shown in FIGS. 1A and 9A, residues 33-666 of SEQ ID NO: 2 correspond to the catalytic domain of clone 19Y Amino acid residues 1 to 596 of SEQ ID NO:4 encompass the catalytic domain of clone 7F.

The full-length native trans-sialidase also has a long 12-amino acid tandem repeat domain in the C-terminus, previously identified as SAPA (i.e., Shed-Acute-Phase-Antigens). Although the tandem repeat is not directly involved in the catalytic activity, it stabilizes the trans-sialidase activity in the blood to increase the half-life of the enzyme from about 7 to about 35 hours. See, Pollevick et al., Mol. Biochem. Parasitol. 47:247 250 (1991) and Buscaglia et al., J. Infect. Dis., 177(2):431 6 (1998) Amino acid residues 667-1162 of SEQ ID NO:4 correspond to the C-terminal tandem repeat of clone 7F. The C-terminal tandem repeat domain is not required for the neurotrophic activity of the PDNF. See, e.g., Chuenkova et al., U.S. Application Publication Nos. 2009/0117593 and 2006/0229247, entire teachings of which are incorporated herein by reference.

A naturally-occurring PDNF may comprise a secretory signal peptide sequence, which causes the transportation of the protein to the T. cruzi surface. A secretory signal peptide sequence is an amino acid sequence that acts to direct the secretion of a mature polypeptide or protein from a cell. Secretory signal peptide sequences are characterized by a core of hydrophobic amino acids and are typically (but not exclusively) found at the amino termini of newly synthesized proteins. When T. cruzi is located extracellularly, PDNF is present both on the parasite outer membrane (Prioli, R. P. et al., Trop. Med. Parasitol., 42:146-150 (1991)) and in the extracellular milieu as a water-soluble, extracellular ligand that binds to cell surface receptors (e.g., TrkA).

Preferably, the PDNF or PDNF fragment of the invention does not comprises a secretory signal peptide sequence so that the PDNF or PDNF fragment remains in the cytoplasm. To retain the PDNF or PDNF fragment in the cytoplasm, the secretory signal peptide sequence of a naturally occurring PDNF can be deleted. For example, FIG. 9E shows a fragment of PDNF (SEQ ID NO: 5) in which the secretory signal peptide sequence (amino acid residues 1-32 of the clone Y19 clone) are deleted. Alternatively, the secretory signal peptide sequence of a naturally occurring PDNF can be mutated so that it is no longer recognized by the secretory pathway of the host cell.

A PDNF fragment comprises a portion, but no the full-length sequence of PDNF, while retaining the anti-apoptotic activity (e.g., retaining the ability to bind to Akt kinase and induce activation of Akt kinase). The PDNF fragments of the invention may or may not have neuraminadase or trans-sialidase catalytic activity as desired. SEQ ID NO:5 is an example of an fragment of PDNF that retains the anti-apoptotic activity. In another exemplary embodiment, the PDNF fragment is residues 1 to 588 of SEQ ID NO: 4. In another exemplary embodiment, the PDNF fragment is residues 1 to 596 of SEQ ID NO: 4.

The PDNF or PDNF fragment of the invention can be a naturally occurring protein which has anti-apoptotic activity (e.g., binds to Akt kinase and activates Akt kinase), or an active variant of a naturally occurring protein.

As used herein, “active variants” refers to variant peptides which retain anti-apoptotic activity (e.g., ability to bind to Akt kinase and induce activation of Akt kinase). An “active variant” may or may not have to have neuraminadase or trans-sialidase catalytic activity as desired. An active variant differs in amino acid sequence from a reference PDNF (such as the PDNF encoded by clone 19Y deposited in GenBank under accession number AJ002174 (SEQ ID NO:2), or the PDNF encoded by clone 7F deposited in GenBank under accession number M61732 (SEQ ID NO:4)), or a reference PDNF fragment (such as SEQ ID NO:5) but retains anti-apoptotic activity (e.g., retains the ability to bind to Akt kinase and induce activation of Akt kinase).

Generally, differences are limited so that the sequences of the reference polypeptide and the active variant are closely similar overall and, in many regions, identical. An active variant of PDNF or PDNF fragment and a reference PDNF or PDNF fragment can differ in amino acid sequence by one or more amino acid substitutions, additions, deletions, truncations, fusions or any combination thereof. Preferably, amino acid substitutions are conservative substitutions. A conservative amino acid substitution refers to the replacement of a first amino acid by a second amino acid that has chemical and/or physical properties (e.g., charge, structure, polarity, hydrophobicity/hydrophilicity) which are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Active variants of PDNF or PDNF fragments include naturally occurring variants (e.g., allelic forms) and variants which are not known to occur naturally.

In one embodiment, an active variant of PDNF shares at least about 85% amino acid sequence similarity or identity with a naturally occurring PDNF (e.g., SEQ ID NO:2, SEQ ID NO:4), preferably at least about 90% amino acid sequence similarity or identity, and more preferably at least about 95%, at least about 96%, at least about 97%, at least about 98%, or at least about 99% amino acid sequence similarity or identity with said PDNF. Preferably, the percentage of identity is calculated over the full length of the active variant.

In certain embodiments, the active variant comprises fewer amino acid residues than a naturally occurring PDNF. In this situation, the variant can share at least about 85% amino acid sequence similarity or identity with a corresponding portion of a naturally occurring PDNF (e.g., SEQ ID NO: 5, or amino acid residues 33-666 of SEQ ID NO:2), preferably at least about 90% amino acid sequence similarity or identity, and more preferably at least about 95%, at least about 96%, at least about 97%, at least about 98%, or at least about 99% amino acid sequence similarity or identity with a corresponding portion of said PDNF.

Portions of the amino acid sequence of PDNF which correspond to a variant and amino acid sequence similarity or identity can be identified using a suitable sequence alignment algorithm, such as ClustalW2 (http://www.ebi.ac.uk/Tools/clustalw2/index.html) or “BLAST 2 Sequences” using default parameters (Tatusova, T. et al., FEMS Microbiol. Lett., 174:187-188 (1999)).

Active variants of PDNF or PDNF fragments can be prepared using suitable methods, for example, by direct synthesis, mutagenesis (e.g., site directed mutagenesis, scanning mutagenesis) and other methods of recombinant DNA technology. Active variants can be identified and/or selected using a suitable assay, such as the co-immunoprecitiation, apoptosis assays, and kinase assays described herein.

Fusion proteins comprising PDNF or a fragment of PDNF are also contemplated. A fusion protein may encompass a polypeptide comprising PDNF (e.g., SEQ ID NO:2, SEQ ID NO:4), a PDNF fragment (SEQ ID NO:5) or an active variant thereof as a first moiety, linked via a covalent bond (e.g., a peptide bond) to a second moiety (a fusion partner) not occurring in PDNF as found in nature. Thus, the second moiety can be an amino acid, oligopeptide or polypeptide. The second moiety can be linked to the first moiety at a suitable position, for example, the N-terminus, the C-terminus or internally. In one embodiment, the fusion protein comprises an affinity ligand (e.g., an enzyme, an antigen, epitope tag, a binding domain) and a linker sequence as the second moiety, and PDNF or a PDNF fragment as the first moiety. Additional (e.g., third, fourth) moieties can be present as appropriate. The second (and additional moieties) can be any amino acid, oligopeptide or polypeptide that does not interfere with the anti-apoptotic activity (e.g., the ability to bind to Akt kinase and induce activation of Akt kinase) of PDNF. Fusion proteins can be prepared using suitable methods, for example, by direct synthesis, recombinant DNA technology, etc.

In certain embodiment, the fusion protein comprises a first moiety which shares at least about 85% sequence similarity or identity with PDNF (e.g., SEQ ID NO:2, SEQ ID NO:4) or a fragment of PDNF (e.g., SEQ ID NO:5), preferably at least about 90% sequence similarity or identity, and more preferably at least about 95%, at least about 96%, at least about 97%, at least about 98%, or at least about 99% sequence similarity or identity with the PDNF or PDNF fragment. Preferably, the percentage of identity is calculated over the full length of the first moiety.

In certain embodiments, a nucleic acid molecule comprising a nucleotide sequence that encodes PDNF, or a fragment of PDNF, is delivered to a cell, so that the PDNF or PDNF fragment is provided as an intracellular protein and binds to Akt kinase. Preferably, the nucleotide sequence encodes PDNF or a fragment of PDNF that lacks a secretory signal peptide sequence.

In one exemplary embodiments, the nucleotide sequence encoding the PDNF or PDNF fragment comprises nucleotides 234 to 2123 of SEQ ID NO: 1. In another exemplary embodiments, the nucleotide sequence encoding the PDNF or PDNF fragment comprises nucleotides 484 to 2248 of SEQ ID NO: 3. In certain embodiments, the nucleotide sequence encoding the PDNF or PDNF fragment is at least 85%, or at least 90%, or at least 95%, or at least 96%, or at least 97%, or at least 98%, or at least 99% identical to nucleotides 234 to 2123 of SEQ ID NO: 1. In certain embodiments, the nucleotide sequence encoding the PDNF or PDNF fragment is at least 85%, at least 90%, at least 95%, or at least 96%, or at least 97%, or at least 98%, or at least 99% identical to nucleotides 484 to 2248 of SEQ ID NO: 3.

B. Cytoplasmic PDNF Induces Activation of Akt Kinase

In one aspect, the PDNF or PDNF fragment of the invention can bind to Akt kinase, and induces activation of Akt kinase (e.g., an increase in the activity level of Akt kinase, an increase in the expression level of a gene encoding Akt kinase, or both).

Akt kinase is a serine-threonine protein kinase that has been implicated in signaling of survival in a wide variety of cells, including fibroblastic, epithelial, and neuronal cells (Franke et al. Cell 1997; 1; 88:435-7; Hemmings et al. Science 1997; 275:628-30). All three mammalian isoforms of Akt (Akt1/PKBα/RAC-PKα, Akt2/PKBβ/RAC-PKβ, and Akt3/RAC-PKγ) have an amino-terminal PH domain, a serine-threonine (S/T) kinase domain related to protein kinase A and C (PKA and PKC) family members, and a carboxy-terminal regulatory domain. Exemplary polynucleotide sequences that encode Akt kinase polypeptides include, e.g., GenBank accession Nos. X65687 (mouse Akt1), U22445 (mouse Akt2), M63167 (human Akt1) M95936 (human Akt2), and AF135794 (human Akt3).

Akt kinase can be activated by multiple mechanisms, including direct binding of phosphoinositides to the pleckstrin homology domain of Akt, and translocation of Akt from the cytoplasm to the nucleus (Datta et al. Genes & Dev 1999; 13:2905-2927).

In certain embodiments, the PDNF or PDNF fragment comprises an Akt phosphorylation site.

In certain embodiments, the phosphorylation of PDNF or a fragment of PDNF results in an increase in Akt kinase activity. In certain embodiments, the phosphorylation of PDNF or a fragment of PDNF results in an increase in the expression level of a gene that encodes Akt kinase.

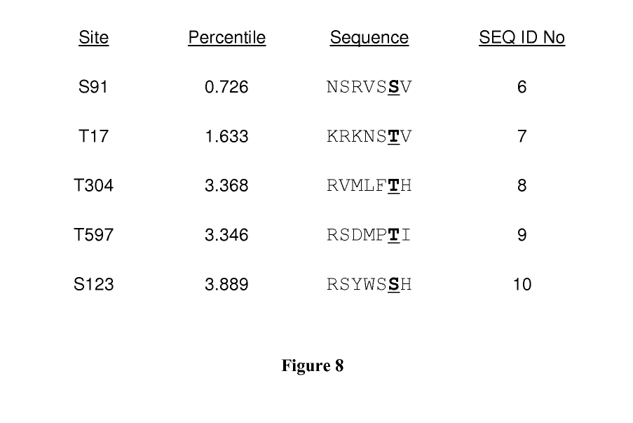

The optimum Akt phosphorylation motif is R—X—R—X—X—S/T-B, wherein X and B represent any amino acid residue and bulky hydrophobic residues, respectively, and S or T represent the phosphorylation targets serine and threonine, respectively. Phosphorylation sites can be predicted using suitable motif scanning/searching programs. For example, using Scansite (http://scansite.mit.edu), SEQ ID NO: 5 (a fragment of PDNF clone 19Y) is predicted to have five sites that could be phosphorylated by Akt (Thr¹⁷, Ser⁹¹, Ser¹²³, Thr³⁹⁴, and Thr⁵⁹⁷) (FIGS. 1A and 8).

Putative phosphorylation sites could be further analyzed based on the structural information of the PDNF. For example, the 3D structure of the N-terminal region of a PDNF isoform reveals a pattern commonly found in other microbial neuraminidases (Gaskell et al., Structure 3:1197-1205 (1995)), having one domain with a α-propeller structure containing catalytic sites for neuraminidase and sialyl transferase activities, and an β-helical segment connecting the β-propeller domain to another domain having a β-barrel lectin-like structure. See, U.S. Application Publication No. 2009/0117593; Buschiazzo et al., Mol. Cell. 10:757-768 (2002). For example, the Thr¹⁷- and Ser⁹¹-containing motifs of SEQ ID NO: 5 have a O-turn and are located on the surface of PDNF (FIG. 1A), thereby readily accessible by Akt kinase. In addition, programs for secondary structure prediction may also be used to analyze the secondary structure or solvent accessibility of PDNF. See, e.g., PHDsec (http://www.embl-heidelberg.de/predictprotein/), NSSP (http://dot.imgen.bcm.tmc.edu: 9331/pssprediction/pssp.html); SOPM: (http://www.ibcp.fr/predict.html); PHDacc (http://www.embl-heidelberg.de/predictprotein/).

In certain embodiments, the phosphorylation site of the PDNF or PDNF fragment of the invention is a serine or threonine residue that corresponds to positions S91, T17, T304, T597, or 5123 of SEQ ID NO:5, or a fragment of SEQ ID NO:5.

In certain embodiments, the phosphorylation site of the PDNF or PDNF fragment of the invention is S91, T17, T304, T597, or 5123 of SEQ ID NO:5, or a fragment of SEQ ID NO:5.

As described and exemplified herein, the phosphorylated PDNF or PDNF fragment induces activation of Akt kinase (e.g., an increase in Akt kinase activity, an increase in the expression level of a gene encoding Akt kinase (including an increase in the mRNA level or the protein level), or both). For example, the mRNA level of Akt kinase in the presence of the phosphorylated PDNF or PDNF fragment can be compared with that in the absence of the phosphorylated PDNF or PDNF fragment. The mRNA level of Akt kinase can be measured and compared using any art known methods, such as microarrays, RT-PCR, northern blot, etc. An increase in the expression level of Akt kinase gene can also be detected by comparing the Akt protein level in the presence of the phosphorylated PDNF or PDNF fragment, relative to that in the absence of the phosphorylated PDNF or PDNF fragment. The protein level of Akt kinase can be measured and compared using any art known methods, such as enzyme-linked immunosorbant assays (ELISA), electrophoretic analysis, Western blots, or radioimmune assays (RIA).

The Akt kinase activity can be measured and compared using any art known methods, including quantification of levels of Akt phosphorylation, quantification of Akt kinase activity, determination of the cellular localization of Akt, quantification of phosphorylation of Akt downstream targets such as mTOR, p70S6 kinase, S6 and GSK-3, and quantification of the kinase activity of Akt downstream targets such as mTOR, p70S6 kinase, and GSK-3.

Akt phosphorylation levels may be quantified, for example, using commercially available antibodies specific for phosphorylated residues of Akt. For example, antibodies specific for human and mouse Akt phosphorylated on residues Ser473, Thr308, Tyr326, or Ser505 are available from a variety of sources, including Biosource International, Covance Research Products, Abcam, Cell Signaling Technology, Novus Biologicals, and R&D Systems. Such antibodies may be used in any of the assays well established in the art, including immunoprecipitation, Western blotting, and ELISA. For example, ELISA kits for quantification of Akt phosphorylated on residues Ser473 or Thr308 are available from a variety of sources, including Biosource International, Cell Signaling Technology, Sigma, and Calbiochem.

Akt kinase activity may be quantified, for example, using an in vitro kinase assay. A variety of Akt kinase assay kits are commercially available, for example, from BioSource International, BioVision, Calbiochem, Cell Signaling Technology, Molecular Devices, Upstate Biotechnology, or Stressgen Biologicals. Peptide substrates of Akt for use in vitro Akt kinase activity assays are commercially available, for example, from BioSource International, Calbiochem, Cell Signaling Technology, and Upstate Biotechnology. Akt kinase assays may be performed as previously described (see, e.g., Nakatani et al. J Biol Chem 1999; 274:21528-21532).

Cellular localization of Akt may be determined by any of the methods well known in the art, e.g. immunocytochemistry using any of the commercially available antibodies to Akt.

Protocols for the quantification of phosphorylation and/or kinase activity of the Akt downstream targets mTOR, p70S6 kinase, S6 and GSK-3 are well established in the art. Phosphorylation of Akt downstream targets such as mTOR, p70S6 kinase, S6 and GSK-3 may be quantitated, for example, using commercially available antibodies. For example antibodies specific for phosphorylated residues of mTOR, p70S6 kinase, S6 or GSK-3 are available from a variety of sources, including Covance Research Products, Abcam, Cell Signaling Technology, Stressgen Bioreagents, Biosource International and Upstate Biotechnology. Such antibodies may be used in any of the assays well established in the art, including immunoprecipitation, Western blotting, and ELISA. ELISA kits for quantification of phosphorylated GSK-3, for example, are available from Active Motif ELISA kits for quantification of phosphorylated p70S6 kinase, for example, are available from R&D Systems. Kinase activity of the Akt downstream targets mTOR, p70S6 kinase, and GSK-3 may be quantified, for example, using an in vitro kinase assay. Such in vitro assays are well described in the art.

C. Cytoplasmic PDNF Induces Suppression of Pro-Apoptotic Activities

In another aspect, the PDNF or PDNF fragment can also induce suppression of pro-apoptotic activities. In particular, the phosphorylated PDNF or PDNF fragment induces a decrease in the activity of a pro-apoptotic protein, a decrease in the expression level of a gene encoding a pro-apoptotic protein (including decreased mRNA level and/or protein level), or both.

As described and exemplified herein, the phosphorylated PDNF or PDNF fragment induces suppression of pro-apoptotic activities mediated by Caspase-9, FOXO, and BAX.

Caspase-9 is an initiator caspase encoded by the CASP9 gene. CASP9 orthologs have been identified in all mammals for which complete genome data are available. Human Caspase-9 is described in Genbank Gene ID No: 842. Caspase-9 is an aspartic acid specific protease and has been linked to the mitochondrial death pathway. It is activated during apoptosis. Induction of stress signaling pathways JNK/SAPK causes release of cytochrome c from mitochondria and activation of apaf-1 (apoptosome), which in turn cleaves the pro-enzyme of caspase-9 into the active form.

FOXO1-FOXO4 (Forkhead box protein O1-O4) belong to the forkhead family of transcription factors which are characterized by a distinct fork head domain. Human FOXO1, FOXO3, and FOXO4 are described in Genbank Gene ID Nos: 2308, 2309, 4303, respectively. Human FOXO3 is also known as human FOXO2. The defining feature of FOX proteins is the forkhead box, a sequence of 80 to 100 amino acids forming a motif that binds to DNA. This forkhead motif is also known as the winged helix due to the butterfly-like appearance of the loops in the protein structure of the domain. Forkhead genes are a subgroup of the helix-turn-helix class of proteins.

The Bc1-2-associated X protein, or Bax is a protein of the Bc1-2 family. It promotes apoptosis by competing with Bc1-2 proper. Human BAX is described in Genbank Gene ID No: 581. Bax is a pro-apoptotic Bc1-2 protein containing BH1, BH2 and BH3 domains. In healthy mammalian cells, the majority of Bax is found in the cytosol, but upon initiation of apoptotic signaling, Bax undergoes a conformation shift, and inserts into organelle membranes, primarily the outer mitochondrial membrane.

The change of mRNA level transcribed by a gene encoding a pro-apoptotic protein, or the protein level of a pro-apoptotic protein can be measured using any art known methods as described above, such as RT-PCR, northern blot, SDS-PAGE, Western blots, immunostaining, or ELISA. The change of activity level of a pro-apoptotic protein can be measured according to the known activity of the protein. For example, caspase-9 antibodies are available (Cell Signaling). Alternatively, the protease activity of caspase-9 can be determined using polypeptides containing a cleavage recognition sequence for caspase-9. Examples of recognition sequences that can be used to measure the activity of caspase-9 can be found in e.g., Thornberry et al., J. Biol. Chem., 272:17907-17911 (1997).

### 4. Methods of Delivery

Modes of delivery of the PDNF or PDNF fragment include direct intracellular delivery of the peptide, and/or administering a nucleic acid molecule encoding the PDNF or PDNF fragment, either in vitro, in vivo or ex vivo.

Methods for direct intracellular delivery of the peptides are known in the art. For example, a polymer-based intracellular delivery system may be used. See, e.g., U.S. Application Publication No. 2004/0101941, which describes intracellular delivery of a protein by conjugating the protein to a polymer such as an N-alkyl acrylamide polymer. Alternatively, cationic lipids can be used for intracellular delivery of a protein (e.g., by encapsulating the protein in a cationic liposome, or associating the protein to form a lipoplex; see e.g., U.S. Application Publication No. 20030008813).

In preferred embodiments, the PDNF or PDNF fragment of the invention is provided by administering to a cell a nucleic acid molecule encoding the PDNF or PDNF fragment. The nucleic acid molecule can be administered directly into a subject (in vivo therapy) or into cells isolated from a subject or a donor (ex vivo therapy). The nucleic acid molecule may be a DNA molecule, an RNA molecule, or may contain a DNA portion, or an RNA portion.

The nucleic acid molecule may be introduced into a cell in several ways. In vitro transfection methods include chemical methods (such as calcium phosphate precipitation and liposome-mediated transfection) and physical methods (such as electroporation). In general, in vitro transfection methods are not suitable for in vivo therapy. For in vivo delivery, the nucleic acid molecules can be delivered either as a non-viral vector, or as a recombinant viral vector such as vectors derived from retrovirus, adenovirus, herpes virus, pox virus, or adeno-associated virus (AAV).

A. Use of Non-Viral Vectors for Nucleic Acid Delivery

Nucleic acid molecules encoding the PDNF or PDNF fragment can be delivered using non-viral vector-based nucleic acid delivery systems, such as the methods as described in U.S. Pat. Nos. 6,413,942, 6,214,804, 5,580,859, 5,589,466, 5,763,270 and 5,693,622. The non-viral vectors may comprise the nucleic acid molecule operably linked to control elements that direct the expression of the coding sequence in a target cell.

Alternatively, non-viral vectors can be packaged in liposomes prior to delivery to a subject or to cells, as described in U.S. Pat. Nos. 5,580,859, 5,549,127, 5,264,618, 5,703,055. For a review of the use of liposomes as carriers for delivery of nucleic acids, see, Hug and Sleight (1991) Biochim. Biophys. Acta. 1097:1-17; Straubinger et al. (1983) in Methods of Enzymology Vol. 101, pp. 512-27; de Lima et al. (2003) Current Medicinal Chemistry, Volume 10(14): 1221-31. The DNA can also be delivered in cochleate lipid compositions similar to those described by Papahadjopoulos et al. (1975) Biochem. Biophys. Acta. 394:483-491. See also U.S. Pat. Nos. 4,663,161 and 4,871,488. For example, a plasmid vector may be complexed with Lipofectamine 2000. Wang et al. (2005) Mol. Therapy 12(2):314-320.

Biolistic delivery systems employing particulate carriers such as gold and tungsten may also be used to deliver non-viral vectors. The particles are coated with the vector and accelerated to high velocity, generally under reduced pressure, using a gun powder discharge from a “gene gun.” See, e.g., U.S. Pat. Nos. 4,945,050, 5,036,006, 5,100,792, 5,179,022, 5,371,015, and 5,478,744.

A wide variety of other methods can be used to deliver the vectors. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, peptoid delivery, or microinjection. See, e.g., Sambrook et al (1989) Molecular Cloning, A Laboratory Manual, Cold Spring Harbor Laboratories, New York, for a discussion of techniques for transforming cells of interest; and Felgner, P. L. (1990) Advanced Drug Delivery Reviews 5:163-87, for a review of delivery systems useful for gene transfer. Exemplary methods of delivering DNA using electroporation are described in U.S. Pat. Nos. 6,132,419; 6,451,002, 6,418,341, 6,233,483, U.S. Patent Publication No. 2002/0146831, and International Publication No. WO 00/45823.

Non-viral vectors may also be introduced directly into the CNS by intrathecal (IT) injection. The vector can be complexed with cationic agents such as polyethyleneimine (PEI) or Lipofectamine 2000 to facilitate uptake. See, e.g., Wang et al. (2005) Mol. Therapy. 12(2):314-320.

B. Use of Viral Vectors for Nucleic Acid Delivery

In certain embodiments, the nucleic acid encoding the PDNF or PDNF fragment of the invention is inserted into a viral vector that is derived from an adenovirus, an adeno-associated virus, a lentivirus, or an alphavirus.

In certain embodiments, replication-deficient viral vectors are preferred.

The adenovirus genome is a linear double-stranded DNA molecule of approximately 36,000 base pairs with the 55-kDa terminal protein covalently bound to the 5′ terminus of each strand. Adenoviral (“Ad”) DNA contains identical Inverted Terminal Repeats (“ITRs”) of about 100 base pairs with the exact length depending on the serotype. The viral origins of replication are located within the LTRs exactly at the genome ends.

Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76).

Use of Adenovirus-derived vectors for gene therapy is known in the art. See, for example, U.S. Pat. No. 6,908,762, U.S. Pat. No. 6,756,226, U.S. Pat. No. 5,824,544; U.S. Pat. No. 5,707,618; U.S. Pat. No. 5,693,509; U.S. Pat. No. 5,670,488; U.S. Pat. No. 5,585,362.

Adenoviral vectors for use with the present invention may be derived from any of the various adenoviral serotypes, including, without limitation, any of the over 40 serotype strains of adenovirus, such as serotypes 2, 5, 12, 40, and 41.

Adeno Associated Virus (AAV) is a parvovirus which belongs to the genus Dependovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation.

The AAV genome is a linear single-stranded DNA molecule containing approximately 4681 nucleotides. The AAV genome generally comprises an internal non-repeating genome flanked on each end by inverted terminal repeats (ITRs). The ITRs are approximately 145 base pairs (bp) in length. The ITRs have multiple functions, including serving as origins of DNA replication and as packaging signals for the viral genome.

AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

Use of AAV-derived vectors for gene therapy is known in the art. See, for example, U.S. Pat. No. 6,489,162, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479.

Retroviruses also provide a convenient platform for gene delivery. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09.

Replication-defective murine retroviral vectors are widely used gene transfer vectors. Murine leukemia retroviruses include a single stranded RNA molecule complexed with a nuclear core protein and polymerase (pol) enzymes, encased by a protein core (gag), and surrounded by a glycoprotein envelope (env) that determines host range. The genomic structure of retroviruses includes gag, pol, and env genes and 5′ and 3′ long terminal repeats (LTRs). Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

Lentivirus is a genus of slow viruses of the Retroviridae family, characterized by a long incubation period. Lentiviruses can deliver a significant amount of genetic information into the DNA of the host cell and have the unique ability among retroviruses of being able to replicate in non-dividing cells, so they are one of the most efficient methods of a gene delivery vector. HIV, SIV, and FIV are all examples of lentiviruses. Use of lentiviral-derived vectors for gene therapy is known in the art. See, for example, U.S. Pat. No. 6,800,281, U.S. Pat. No. 6,277,633.

Additional viral vectors useful for delivering the nucleic acid molecules include those derived from the pox family of viruses, including vaccinia virus and avian poxvirus.

Avipoxviruses, such as the fowlpox and canarypox viruses, can be used to deliver the genes. Recombinant avipox viruses expressing immunogens from mammalian pathogens are known to confer protective immunity when administered to non-avian species. The use of avipox vectors in human and other mammalian species is advantageous with regard to safety because members of the avipox genus can only productively replicate in susceptible avian species. Methods for producing recombinant avipoxviruses are known in the art and employ genetic recombination, as described above with respect to the production of vaccinia viruses. See, e.g., WO 91/12882; WO 89/03429; and WO 92/03545.

The nucleic acid molecule may also be delivered using an alphavirus-derived vector. Many properties of alphavirus vectors make them a desirable alternative to other virus-derived nucleic acid delivery systems being developed, including the ability to (i) rapidly engineer expression constructs, (ii) produce high-titered stocks of infectious particles, (iii) infect non-dividing cells, and (iv) attain high levels of expression (Strauss and Strauss, Microbiol. Rev. 1994, 58:491-562; Liljestrom et al., Biotechnology 1991, 9:1356-1361; Bredenbeek et al., Semin. Virol. 1992, 3:297-310; Xiong et al., Science 1993, 243:1188-1191). Defective Sindbis viral vectors have been used to protect mammals from protozoan parasites, helminth parasites, ectoparasites, fungi, bacteria, and viruses (PCT Publication No. WO 94/17813).

A cDNA encoding Venezuelan Equine Encephalitis (VEE) and methods of preparing attenuated Togaviruses have been described (U.S. Pat. No. 5,185,440). Infectious Sindbis virus vectors have been prepared with heterologous sequences inserted into the structural region of the genome (U.S. Pat. No. 5,217,879). In addition, RNA vectors based on the Sindbis Defective Interfering (DI) particles with heterologous sequences have also been described (U.S. Pat. No. 5,091,309). Alphaviruses, specifically the Semliki Forest Virus, were used medically to deliver exogenous RNA encoding heterologous genes, e.g., an antigenic epitope or determinant (PCT Publications No. WO 95/27069 and WO 95/07994). Vectors for enhanced expression of heterologous sequences downstream from an alphavirus base sequence have been also disclosed (PCT Publication No. WO 95/31565).

Sindbis virus is a member of the alphavirus genus and has been studied extensively since its discovery in various parts of the world beginning in 1953 (see Taylor et al., Egypt. Med. Assoc., 1953, 36:489-494; Taylor et al., Am. J. Trop. Med. Hyg, 1955, 4:844-862; and Shah et al., Ind. J. Med. Res, 1960, 48:300-308). Like many other alphaviruses, Sindbis virus is transmitted to vertebrate hosts from mosquitos. Alphavirus virions consist of a nucleocapsid, wrapped inside a lipid bilayer, upon which the envelope proteins are displayed. The envelope proteins mediate binding to host cell receptors, leading to the endocytosis of the virion. Upon endocytosis, the nucleocapsid, a complex of the capsid protein and the genomic viral RNA, is deposited into the cytoplasm of the host cell. The Sindbis virus genome is a single-stranded 49S RNA of 11703 nt (Strauss et al., 1984, Virology, 133: 92-110), capped at the 5′ terminus and polyadenylated at the 3′ terminus. The genomic RNA is of (+)-sense, is infectious, and serves as mRNA in the infected cell. Translation of the genomic RNA gives rise to the nonstructural proteins, nsP1, nsP2, nsP3, and nsP4, which are produced as polyproteins and are proteolytically processed. Early during infection, the nonstructural proteins, perhaps in association with host factors, use the genomic (+)-sense RNA as template to make a full-length, complementary (−) strand RNA. The (−) strand is template for synthesis of full-length genomic RNA. An internal promoter on the (−) strand is used for transcription of a subgenomic 26S mRNA which is co-linear with the 3′ terminal one-third of the genomic RNA. This 26S subgenomic mRNA is translated to produce a structural polyprotein that undergoes co-translational and post-translational cleavages to produce the structural proteins: C (capsid), E2, and E1 (envelope). The capsid protein C encapsidates the genomic RNA to form nucleocapsids. These interact with the cytoplasmic domain of the cell surface-bound viral envelope proteins, resulting in the envelopment of the nucleocapsid inside a membrane bilayer containing the envelope proteins, and the budding of progeny virions out of the infected cell. Sindbis virus infection has been shown to induce apoptosis in a host cell (Levine et al., Nature, 1993, 361; 739-742; Jan AND GRIFFIN, J. Virol., 1999, 73:10296-10302).

C. Expression Control Sequences

In certain embodiments, the nucleic acid molecule is a vector comprising a nucleotide sequence that encodes PDNF or PDNF fragment that is operably linked to an expression control sequence that promotes the expression of the PDNF or PDNF fragment in a mammalian cell. The expression control sequence may be a promoter, an enhancer, a ribosome entry site, or a polyadenylation sequence, for example.

For example, an inducible promoter may be used to control the expression of the PDNF or PDNF fragment. For example, a tetracycline responsive promoter has been used effectively to regulate transgene expression in rat brain (Mitchell & Habermann, 1999 Biol Res Nurs 1:12-19). Other inducible promoters include hormone-inducible promoters (No et al., Proc Natl Acad Sci USA (1996) 93:3346-51.; Abruzzese et al., Hum Gene Ther (1999) 10:1499-1507.; Burcin et al., Proc Natl Acad Sci USA (1999) 96:355-360), radiation-inducible promoters, such as those employing the Egr-1 promoter or NF-.quadrature.B promoter (Weichselbaum et al., J Natl Cancer Inst (1991) 83:480-484.; Weichselbaum et al., Int J Radiat Oncol Biol Phys (1994) 30:229-234), and heat-inducible promoters (Madio et al., J Magn Reson Imaging (1998) 8:101-104.; Gerner et al., Int J Hyperthermia (2000) 16:171-181.; Vekris et al., J Gene Med (2000) 2:89-96). Promoters contemplated by the invention include, but are not limited to, ubiquitous promoters such as the cytomegalovirus (CMV) promoter/enhancer, long terminal repeat (LTR) of retroviruses, the γ-actin promoter and the like, tissue specific promoters such as Tie-2, VE-cadherin and other endothelial cell and bone marrow-specific promoters, and inducible promoters such as the tetracycline (Tet) promoter.

Other expression control sequences contemplated for use in the invention include enhancers, introns, polyadenylation signal, and 3′UTR sequences.

### 5. Pharmaceutical Compositions and Methods of Administration

In another aspect, the invention relates to the PDNF or PDNF fragment as described herein for use in therapy (e.g., for treating a neurodegenerative disease). In certain embodiments, the nucleic acid molecules as described herein are administered to a mammalian subject in need of reducing cell apoptosis. In certain embodiments, the mammalian subject is a human. For example, the subject may be suffering from or susceptible to a neurodegenerative disease. Alternatively or in addition, the subject may be in need of reducing apoptosis of neurons or glial cells (such as Schwann cells). Treatment a disease includes preventing or delaying the onset or progression of the diseases, mitigating the severity of the diseases, or protecting the cells from further damages, or ameliorating symptoms. Treatment also includes prophylactic treatment of a subject that has not manifested a disease phenotype.

In another aspect, the invention provides a method of reducing the effect of an apoptotic-inducing agent on a mammalian subject, by administering a nucleic acid molecule comprising a nucleotide sequence that encodes PDNF, or a fragment of PDNF, as described herein. An apoptotic-inducing agent can be a variety of different insults to the cell including, molecular, environmental and physical stimuli. Such stimuli are known to those skilled in the art and can be characterized by activating a molecule within the apoptotic pathway. Examples of apoptotic-inducing agents include inducers such as deprivation of a growth factor, pro-inflammatory cytokines, free radicals, oxidative stress, Fas ligand, anti-Fas antibody, staurosporine, Tumor Necrosis Factor, ultraviolet and gamma-irradiation.

For example, pro-inflammatory cytokines (e.g., TGF-β or TNF-α), H₂O₂, and free radicals can cause apoptosis. The increased incidence of neurodegenerative diseases may be attributed to a pro-oxidative environment caused by smoking, alcohol abuse, UVA and UVB radiations, air pollution as well as inappropriate nutrition.

The generation of oxygen-free radicals by the addition of Fe²⁺ to cells has been reported. Generation of oxygen free radicals has been implicated in neurodegeneration, and evidence of oxidative stress has been shown in Alzheimer's disease brain, where oxidative stress contributes to the formation of amyloid plaques and neurofibrillary. β-amyloid is a component of Alzheimer's disease plaques and can cause increases in reactive oxygen species (ROS) via several mechanisms. The harmful effect of oxidative stress can be mitigated by the administration of PDNF or PDNF fragments that function intracellular to activate Akt kinase.

In another aspect, the invention provides a pharmaceutical composition comprising a nucleic acid molecule comprising a nucleotide sequence that encodes PDNF, or a fragment of PDNF, as described herein.

The pharmaceutical composition may further comprise one or more pharmaceutically acceptable carriers, diluents, or excipients. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Poloxamer (Pluronic F68), any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

One particularly useful formulation comprises the vector in combination with one or more dihydric or polyhydric alcohols, and, optionally, a detergent, such as a sorbitan ester. See, e.g., International Publication No. WO 00/32233.

One of skill in the art should be able determine an effective dose empirically. Methods of determining the most effective means and dosages of administration are well known to those of skill in the art and will vary with the pharmaceutical composition, the target cells, and the subject being treated. Administration can be effected in one dose, continuously or intermittently throughout the course of treatment. Single and multiple administrations can be carried out with the dose level and pattern being selected by the treating physician or researcher.

An “effective amount” or “therapeutically effective amount” is an amount that is sufficient to achieve the desired therapeutic or prophylactic effect, such as an amount sufficient to reduce/ameliorate symptoms of a disease that is associated with apoptosis (e.g., neurondegeneration, cognitive decline, impairment of movement, etc), prevent or delay the onset or progression of the disease, mitigate the severity of the disease, or protect the cells from further damages.

The dosage administered, as single or multiple doses, to an individual will vary depending upon a variety of factors, including pharmacokinetic properties, the route of administration, patient conditions and characteristics (sex, age, body weight, health, size), extent of symptoms, concurrent treatments, frequency of treatment and the effect desired.

If the PDNF or PDNF fragment is administered directly as a protein, an effective amount of a pharmaceutical formulation will deliver a dose of about 0.001 to about 100 mg/kg body weight, about 0.01 to about 100 mg/kg body weight, about 0.01 to about 10 mg/kg body weight, about 0.01 to about 5 mg/kg body weight, about 0.01 to about 3 mg/kg body weight, about 0.01 to about 2 mg/kg body weight, about 0.01 to about 1 mg/kg body weight, about 0.1 to about 100 mg/kg body weight, about 0.1 to about 10 mg/kg body weight, about 0.1 to about 5 mg/kg body weight, about 0.1 to about 3 mg/kg body weight, about 0.1 to about 2 mg/kg body weight, about 0.1 to about 1 mg/kg body weight, about 0.01 to about 1000 μg/kg body weight, about 0.01 to about 100 μg/kg body weight, about 0.1 to about 100 μg/kg body weight, about 1 to about 100 μg/kg of body weight, about 1 to about 50 μg/kg body weight, about 5 to about 50 μg/kg body weight, or about 10 to about 50 μg/kg body weight. For example, the dosage may be about 0.02 mg/kg body weight, about 0.25 mg/kg body weight, about 0.5 mg/kg body weight, about 0.75 mg/kg body weight, about 1 mg/kg body weight, about 2 mg/kg body weight, etc.

If a nucleic acid molecule encoding the PDNF or PDNF fragment is administered, an effective amount of a pharmaceutical formulation will deliver a dose of from about 10 ng to about 1 g, about 10 ng to about 100 mg, from about 100 ng to about 100 mg, from about 1 μg to about 100 mg, from about 1 μg to about 10 mg, from about 1 μg to about 5 mg, from about 1 μg to about 1 mg, from about 1 μg to about 0.5 mg, from about 1 μg to about 0.25 mg, from about 5 μg to about 0.5 mg, or from about 25 μg to about 0.5 mg, nucleic acid per patient. Doses for viral vectors may vary from about 1 to about 10000, from about 1 to about 1000, from about 10 to about 1000, from about 10 to about 100, from about 10 to about 50 virions per dose.

Dosage can be by a single dose schedule or a multiple dose schedule. In a multiple dose schedule the various doses may be given by the same or different routes. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, about 6 months, about 9 months, about 1 year, about 2 years etc.).

The pharmaceutical composition may be formulated into compositions for CNS or peripheral nervous system delivery. Oral (including buccal and sub-lingual), rectal, nasal, topical, pulmonary, vaginal or parenteral (including intramuscular, intraarterial, intrathecal, subcutaneous and intravenous) administration or administration by inhalation or insufflation are also contemplated. Common target areas for neural tissue administration include peripheral nerves, the retina, dorsal root ganglia, neuromuscular junction, as well as the CNS, e.g., to target spinal cord glial or striatum cells.

Intrathecal administration overcomes the blood-brain barrier (BBB) by direct injection into the cerebrospinal fluid. Intrathecal administration is described in greater detail with reference to administration of therapeutic vectors.

Intranasal delivery (ND) is a noninvasive alternative method of bypassing the BBB to deliver therapeutic agents to the brain and spinal cord, eliminating the need for systemic delivery and thereby reducing unwanted systemic side effects. ND works because of the unique connection between the nerves involved in sensing odors and the external environment. Delivery from the nose to the central nervous system takes place within minutes along both the olfactory and trigeminal neural pathways. Delivery occurs by an extracellular route and does not require that the drugs bind to any receptor or undergo axonal transport. Bulk flow through perivascular and hemangiolymphatic channels may also be involved in the movement of drugs from the nose to the brain and spinal cord. The precise mechanism of ND is not an important element of the invention.

Recombinant vectors may be introduced into any neural tissue including, without limitation, peripheral nerves, retina, dorsal root ganglia, neuromuscular junction, as well as the CNS. Recombinant vectors of the present invention can be delivered using either ex vivo or in vivo transduction/transfection techniques.

For ex vivo delivery, the desired recipient cell is removed from the subject, transduced/transfected with vector in vitro, formulated into a pharmaceutical composition and reintroduced into the subject in one or more doses. In some embodiments, recipient cells harboring the nucleic acid molecules are screened using conventional techniques such as Southern blots and/or PCR, or by using selectable markers, prior to reintroduction into the subject. Alternatively, syngeneic or xenogeneic cells can be used for ex vivo therapy, provided that they will not generate an undesired immune response in the subject. Neural progenitor cells may also be transduced in vitro and then delivered to the CNS.

For in vivo delivery, recombinant vectors are formulated into pharmaceutical compositions and one or more doses are administered. Therapeutically effective doses can be readily determined by one of skill in the art and will depend on the particular delivery system used.

Recombinant vectors, or cells transduced/transfected in vitro, may be delivered directly to neural tissue by injection into the ventricular region, the striatum (e.g., the caudate nucleus or putamen of the striatum), the spinal cord or a neuromuscular junction with a needle, catheter or related device, using neurosurgical techniques known in the art, such as, where appropriate, by stereotactic injection. See, e.g., Stein et al. (1999) J. Virol. 73:3424-29; Davidson et al. (2000) Proc. Natl. Acad. Sci. (USA) 97:3428-32; Davidson et al. (1993) Nat. Genet. 3:219-23; and Alisky and Davidson (2000) Hum. Gene Ther. 11:2315-29.

One method for targeting the CNS is by intrathecal delivery. Intrathecal delivery is effected by delivering a therapeutic substance to the cerebrospinal fluid (CSF) in the intrathecal (subarachnoid) space, located between the arachnoid membrane and the pia mater, which adheres to the surface of the spinal cord and brain. Delivery to the intrathecal space bypasses the blood brain barrier (BBB), allowing for accumulation of a therapeutic substance within the CNS. The BBB also serves to prevent leaking of relatively impermeable substances into general circulation, thus avoiding systemic side effects that might otherwise occur.

Intrathecal injection is typically made at either the L3/L4 or L4/L5 intervertebral space in adult human subjects, or L4/5 or L5/S1 for infants. Because post-administration complications such as headache are associated with larger bore needles for intrathecal delivery, a small bore needle should be used, e.g. a 22-25 gauge pencil-point needle, e.g. Whitacre G27 (Becton-Dickinson, Rutherford, N.J.). Intrathecal delivery can be via bolus injection, which can optionally be repeated, or by continuous infusion using a surgically implanted catheter and pump (e.g. an osmotic pump). Commercially available systems for intrathecal delivery include the SynchroMed® EL and SynchroMed® II intrathecal drug delivery systems (Medtronic, Minneapolis, Minn.). The details of intrathecal administration procedure, however, will be determined by a researcher or medical practitioner in light of the subject at issue, and is not a crucial aspect of the present invention.

Another method for administering recombinant vectors or transduced/transfected cells is by delivery to dorsal root ganglia (DRG) neurons, e.g., by injection into the epidural space with subsequent diffusion to DRG. For example, recombinant vectors or transduced cells can be delivered via intrathecal cannulation under conditions where the protein diffuses to DRG. See Chiang (2000) Acta Anaesthesiol. Sin. 38:31-36; Jain (2000) Expert Opin. Investig. Drugs 9:2403-10.

Yet another mode of administration to the CNS uses convection-enhanced delivery (CED). Bobo et al. (1994) Proc. Nat'l Acad. Sci. (USA) 91:2076-80. In this way, recombinant vectors can be delivered to many cells over large areas of the CNS. Moreover, the delivered vectors efficiently express transgenes in CNS cells (e.g., glial cells). Any convection-enhanced delivery device may be appropriate for delivery of recombinant vectors. In a preferred embodiment, the device is an osmotic pump or an infusion pump. Both osmotic and infusion pumps are commercially available from a variety of suppliers, for example Alzet Corporation, Hamilton Corporation, Alza, Inc. Typically, a recombinant vector is delivered via CED devices as follows. A catheter, cannula or other injection device is inserted into CNS tissue in the chosen subject. Stereotactic maps and positioning devices are available, for example from ASI Instruments (Warren, Mich.). Positioning may also be conducted by using anatomical maps obtained by CT and/or MRI imaging to help guide the injection device to the chosen target. Moreover, because the methods described herein can be practiced such that relatively large areas of the subject take up the recombinant vectors, fewer infusion cannula are needed. Since surgical complications are related to the number of penetrations, this mode of delivery serves to reduce the side-effects seen with conventional delivery techniques. For a detailed description regarding CED delivery, see U.S. Pat. No. 6,309,634.

Therapeutic vectors may also be administered intranasally, or parenterally (including intramuscular, intraarterial, subcutaneous and intravenous).

### Exemplification

The invention now being generally described, it will be more readily understood by reference to the following examples, which are included merely for purposes of illustration of certain aspects and embodiments of the present invention, and are not intended to limit the invention.

### Example 1

### Anti-Apoptotic Effect of Cytoplasmic PDNF

In this example, we show that in the cytosol, parasite-derived neurotrophic factor (PDNF), a trans-sialidase that is located on the surface of T. cruzi, is a substrate and an activator of the serine-threonine kinase Akt, a prototypic anti-apoptotic molecule. PDNF was shown to increase the expression of the gene that encodes Akt while suppressing the transcription of genes that encode pro-apoptotic factors. Consequently, PDNF elicited a sustained functional response that protects host cells from apoptosis induced by oxidative stress and the pro-inflammatory cytokines tumor necrosis factor-α and transforming growth factor-β. Given that PDNF was also shown to activate Aid by binding to the neurotrophic surface receptor TrkA, we propose that this protein activates survival signaling both at the cell surface by acting as a receptor-binding ligand and inside cells, downstream of the receptor, by acting as a scaffolding adaptor protein.

1. Introduction

The parasite Trypanosoma cruzi, which causes Chagas' disease, differentiates in the cytosol of its host cell and then replicates and spreads infection, processes that require the long-term survival of the infected cells.

Chagas' disease can afflict patients for many years or even decades and commonly starts when the obligate intracellular parasite Trypanosoma cruzi gains access to cells in the skin or in the mucosa after release from reduviid insect excreta. T. cruzi binds to receptors on the surface of host cells, which leads to its internalization in phagolysomes. It then escapes to the cytosol where it differentiates, replicates, grows, and spreads the infection to neighboring cells through the extracellular matrix and to distant cells through the circulation (1, 2). T. cruzi also uses the cell cytosol as reservoir, as exemplified by the infection of adipose tissue in the murine model of Chagas' disease (3). The crosstalk between T. cruzi and components of the host cytosol is critical for survival of the parasite and the dissemination and maintenance of infection in mammalian hosts; however, the molecular basis underlying the interaction of the parasite with the intracellular milieu remains largely unexplored. For example, little, if anything, is known about why cells stay alive for so long while harboring a large number of trypanosomes that require space, nutrients, and other host-cell factors for proper intracellular parasitism.

We have shown that the glycophosphoinositol (GPI)-anchored parasite-derived neurotrophic factor (PDNF) of T. cruzi, known mostly for its neuraminidase (4) and sialyl-transferase (5) activities, binds to the receptor tyrosine kinases TrkA and TrkC (6, 7). These receptors are typically activated after engagement with the neurotrophins nerve growth factor (NGF) and neurotrophin-3 (NT-3) during development and the repair of the nervous system (8). Neurotrophin-Trk receptor interactions activate downstream signaling cascades, including the phosphatidylinositol 3-kinase (PI3K)-Akt kinase pathway, which enchances cell survival, proliferation, and size, as well as protein synthesis, response to nutrient availability, and other activities that are important for cellular survival and homeostasis (9, 10). Underscoring its mimicry of neurotrophins, the binding of PDNF to TrkA and TrkC induces the survival and differentiation of neurons and Schwann cells (6, 7, 11). Uniquely, the recognition of TrkA by T. cruzi promotes cellular invasion (12). These actions require the activation of downstream signaling pathways, including the PI3K-Akt kinase pathway (6, 7). It is thought that the activation of Trk-dependent PI3K-Akt signaling by T. cruzi is important for the survival of infected cells (6, 7, 12). The interactions between T. cruzi and Trks and other cell surface receptors last for only minutes and, thus, cannot solely account for the protection against the damaging events that result from long-lasting intracellular parasitism. However, host cell defense must be an important factor that enables T. cruzi to establish chronic infection despite a strong immune response to the parasite (13).

PDNF is anchored to the surface of T. cruzi by a GPI linkage (14) and shed into the environment, including the cell cytosol (14-17), such that cytoplasmic PDNF is readily available to interact with Akt and other cytoplasmic signaling factors. Here, we show that Akt phosphorylates PDNF, which in turn activates Akt, increases the expression of the gene that encodes Aid, and inhibits the expression of genes that encode pro-apoptotic proteins. Consequently, T. cruzi-infected and PDNF-transfected cells strongly resist the potent pro-apoptotic stimuli tumor necrosis factor α (TNF-α) and transforming growth factor-β (TGF-β) and oxidative stress induced by hydrogen peroxide (H₂O₂). PDNF and activated Akt are most abundant late in the T. cruzi intracellular cycle, when the parasite burden is maximal. Thus, the targeting of Akt by T. cruzi could be an important mechanism that underlies the long-term survival of infected cells

2. PDNF is a Substrate of the Ser-Thr Kinase Akt

We used a combination of bioinformatics, immunochemistry, intracellular colocalization microscopy, and in vitro enzymatic approaches to address the question of whether PDNF is a substrate of the Ser-Thr kinase Akt (also known as protein kinase B (PKB)). The optimum Akt phosphorylation motif is R—X—R—X—X—S/T-B (SEQ ID NO:15), where X and B represent any amino acid residue and bulky hydrophobic residues, respectively, and S or T represent the phosphorylation targets serine and threonine, respectively (18). Scanning the PDNF clone 19Y, which consists of a N-terminal region of 632 amino acid residues that contains the trans-sialidase catalytic domain and a C-terminal region composed of a tandem repeat unit of 12 amino acid residues (D-S—S-A-N-G-T-P—S-T-P-A) (SEQ ID NO:13) (19, 20), the motif-searching program Scansite (http://scansite.mit.edu) (21) predicted the presence of five sites that could be phosphorylated by Akt (Thr¹⁷, Ser⁹¹, Ser¹²³, Thr³⁰⁴, and Thr⁵⁹⁷) (FIGS. 1A and 8) (20). The Thr¹⁷- and Ser⁹¹-containing motifs have a β-turn and are located on the surface of PDNF (FIG. 1A) (22). Thus, the phosphorylation motifs of PDNF should be readily accessible to Akt if it were to interact with T. cruzi.

A physical interaction between PDNF and Akt was demonstrated by coimmunoprecipitation (IP) assays. We infected Schwann cells with T. cruzi for 2 hours, removed parasites from the medium overlay by washing, and cultured the cells for 4 days to allow intracellular parasites to differentiate, grow, and multiply. Lysates of uninfected and infected Schwann cells were immunoprecipitated with the monoclonal antibody (mAb) TCN-2, which is specific for the C-terminal tandem repeat unit of PDNF (FIG. 1A) (23), and Western blots of these immunoprecipitates were incubated with PDNF— and Akt-specific antibodies. The results showed that TCN-2 coimmunoprecipitated PDNF and Akt from the lysates of T. cruzi-infected human Schwann cells, but not from uninfected Schwann cells (FIG. 1B).

In addition, immunoprecipitated PDNF was detected with an antibody that was specific for degenerate phosphopeptides of the sequence R—X—R—X—X-pT/S (SEQ ID NO:14) and thus could detect phosphorylated substrates of Akt (21, 24) (FIG. 1C). Converse immunoprecipitation assays showed that proteins coimmunoprecipitated by the antibody specific for phosphorylated substrates of Akt were readily detected with the PDNF-specific antibody TCN-2 (FIG. 1C, middle panel) and migrated in SDS-polyacrylamide gel electrophoresis (SDS-PAGE) gels similarly to PDNF (FIG. 1C, right panel). Thus, it seemed that Akt phosphorylated PDNF in T. cruzi-infected Schwann cells, a conclusion consistent with fluorescence microscopy analysis that showed a profound increase in the amount of Akt-phosphorylated substrate in Schwann cells that bore cytosolic parasites. In the fluorescence microscopy analysis, uninfected and T. cruzi-infected Schwann cells were incubated with an antibody against Akt-phosphorylated substrates, the anti-PDNF mAb TCN-2, and DAPI, respectively. Images of (1) Akt-phosphorylated substrate staining and (2) PDNF staining showed identical patterns. This confirms that PDNF colocalized with an Akt-phosphorylated substrate on the surface of intracellular T. cruzi trypomastigotes in the infected Schwann cells.

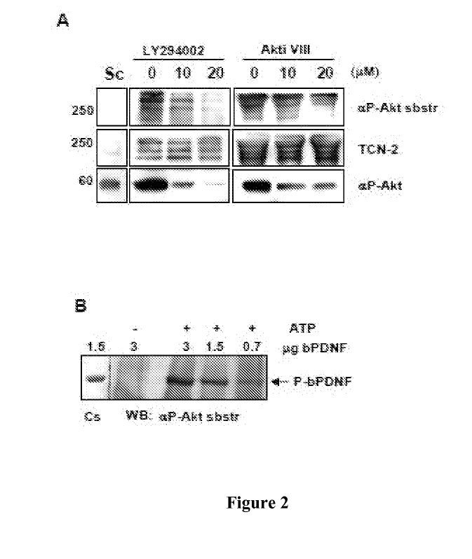

To provide further evidence that the phosphorylation of PDNF depended on host Aid, T. cruzi-infected Schwann cells were treated with LY294002, a pharmacological inhibitor of PI3K, which phosphorylates and activates Akt (10), and with Akti VIII, a specific allosteric inhibitor of Akt that prevents the phosphorylation of Akt at Ser⁴⁷³ and Thr³⁰⁸, which is critical for the activation of Akt (25). Lysates of treated cells were assayed for phosphorylated Akt (pAkt) and, after immunoprecipitation with a PDNF-specific antibody, for the presence of Akt-phosphorylated substrates. The inhibition of the phosphorylation of PDNF corresponded with a reduction in abundance of activated Akt (FIG. 2A). The block in the formation of phospho-PDNF was specific, because inhibitors of the activation of Akt did not affect the abundance of unphosphorylated PDNF (FIG. 2A). The conclusion that PDNF was a substrate of Akt was further reinforced by in vitro kinase assays. Mixing unphosphorylated recombinant PDNF isolated from Escherichia coli (bacterial PDNF, bPDNF) (26) with purified, activated Akt generated phosphorylated PDNF in vitro in a dose-dependent and ATP-dependent manner (FIG. 2B).

3. Developmental Regulation of the Activation of Akt in T. Cruzi-Infected Schwann Cells

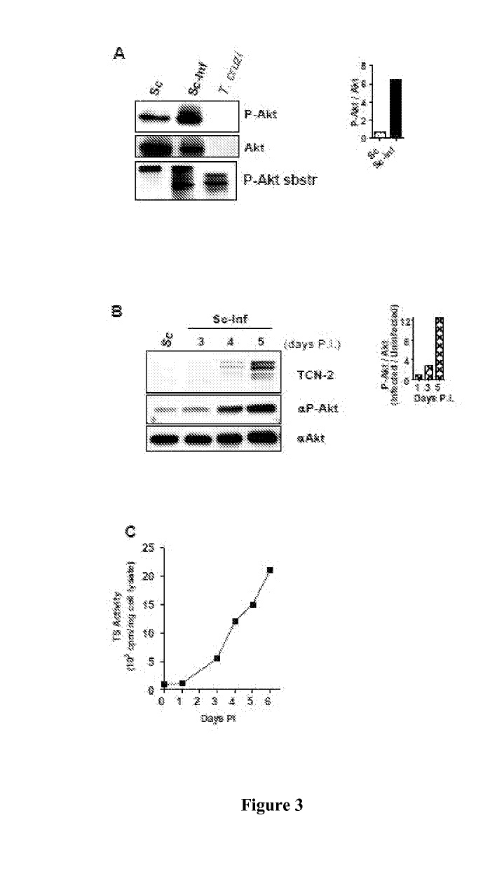

To determine whether intracellular T. cruzi activated Aid, we examined lysates of uninfected and 4- to 5-day infected Schwann cells by Western blotting with antibodies specific for Akt phosphorylated at Ser⁴⁷³, a marker of the activation of Akt (10), in parallel with antibodies specific for Akt and phosphorylated substrates of Akt. These experiments showed that the abundance of pAkt increased by 6-fold in T. cruzi-infected cells compared to that in uninfected cells (FIG. 3A), which indicated the robust activation of Akt in infected cells. This was accompanied by a substantial increase in the formation of phosphorylated substrates of Akt (FIG. 3A, lower panel), which indicated that the activated Akt was functional.

Because Schwann cells were filled with T. cruzi 4 to 5 days post-infection (PI), the observed increase in the abundance of pAkt could be an artifact caused by a cross-reaction of the Akt-specific antibodies with an Akt-like enzyme from T. cruzi. However, this possibility could be excluded because the antibodies specific for Akt and for pAkt did not react with purified T. cruzi trypomastigotes (FIG. 3A). In contrast, the antibody specific for phosphorylated substrates of Akt bound to the purified parasites (FIG. 3A). This finding further confirmed that Akt phosphorylated T. cruzi and that the parasites bore Akt-phosphorylated substrates such as PDNF/trans-sialidase.

To determine whether the activation of Akt by T. cruzi was developmentally regulated, we incubated lysates of Schwann cells infected with T. cruzi for 3, 4, or 5 days with antibodies specific for PDNF, activated Aid, and total Aid and assayed them for trans-sialidase enzymatic activity, a widely-studied property of PDNF (5). Confirming earlier results (4, 16), we found that the production of PDNF, as determined by Western blotting and trans-sialidase activity assays, increased in line with the progression of infection (FIG. 3, B and C). This increase correlated with the formation of pAkt, which was >10-fold more abundant in cells 4 days post-infection (PI) than in uninfected cells (FIG. 3B).

### Cytosolic PDNF Activates Akt, Increases Expression of the Gene Encoding Akt, and Inhibits the Expression of Genes Encoding Pro-Apoptotic Factors

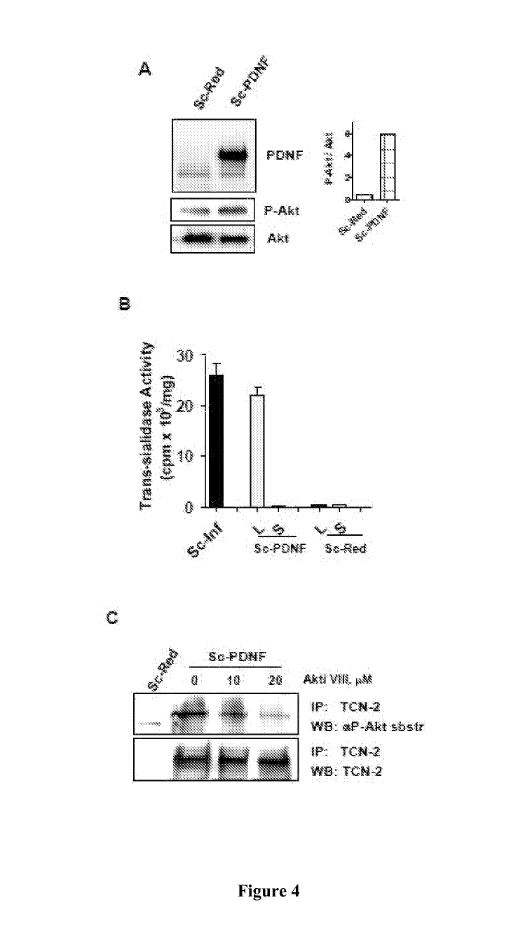

To validate the Akt-dependent phosphorylation of PDNF, we subcloned the full-length cDNA of the gene encoding PDNF into the mammalian pIRES2-DsRed-Express bicistronic expression vector. Schwann cells were then transfected with either empty vector or with the plasmid containing the PDNF gene. Schwann cell-expressed PDNF caused a five-fold increase in the abundance of pAkt (Ser⁴⁷³) and a six-fold increase in the abundance of pAkt (Thr³⁰⁸) compared to that in control transfected cells (FIG. 4A and inset), confirming that the increased activation of Akt in T. cruzi-infected cells was, at least in part, triggered by PDNF. The increase in the activation of Akt was not caused by leakage of PDNF into the culture supernatants (FIG. 4B), which otherwise might activate Akt through its binding to TrkC receptors on the surface of Schwann cells. The abundance of PDNF in transfected Schwann cells was comparable to that in T. cruzi-infected cells (4 days PI), as determined by measurement of the intrinsic trans-sialidase activity of PDNF (FIG. 4B). Furthermore, precipitating PDNF from the lysates of Schwann cells containing PDNF with the monoclonal antibody TCN-2 and examining the immunoprecipitates for the presence of phosphorylated substrates of Akt corroborated the results from the T. cruzi-infected Schwann cells, namely, that the detection of phosphorylated PDNF with the antibody specific for phosphorylated substrates of Akt was dependent on Aid, because the specific Akt inhibitor VIII substantially decreased the extent of the phosphorylation of PDNF (FIG. 4C).

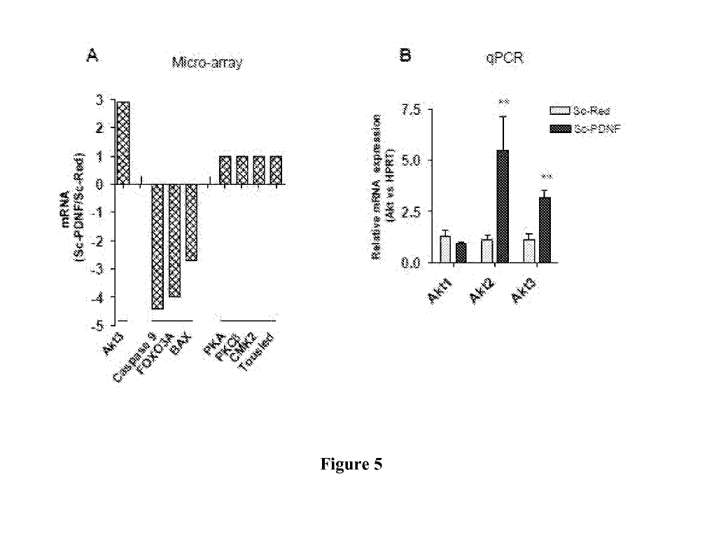

In addition to the enhanced activation of Akt, we found that cytosolic PDNF increased the expression of the gene encoding Akt in Schwann cells as determined by cDNA microarray and real-time polymerase chain reaction (PCR) assays. The results showed that the messenger RNA (mRNA) for isoform 3 of Akt (Akt3) was ˜3-fold more abundant in Schwann cells expressing PDNF than in control cells, whereas the abundance of the mRNAs for other protein kinases were unaltered (FIG. 5A). In contrast, mRNAs for the pro-apoptotic proteins caspase-9, the transcription factor FOXO, and the mitochondrial protein BAX were reduced in abundance by ˜5.0-, ˜4.0- and ˜2.6-fold, respectively, in PDNF-expressing Schwann cells compared to those in control cells (FIG. 5A). Real-time PCR confirmed the 3-fold increased expression of the gene encoding Akt3 in the PDNF-transfected Schwann cells compared to that in control cells. In addition, these experiments showed an increase in the mRNA for Akt2 by ˜5.0-fold (p<0.05) in PDNF-expressin cells compared to that in control cells, but in the Akt1 mRNA (FIG. 5B).

Schwann Cells Bearing Cytosolic T. cruzi or PDNF Strongly Resist Exogenous Apoptotic Stimuli

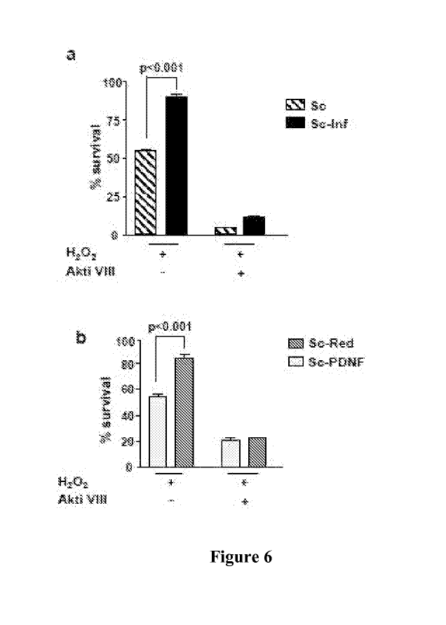

The combination of the activation of Akt, the increased expression of the gene encoding Aid, and the decreased expression of genes encoding pro-apoptotic factors could represent a strategy by T. cruzi to counter host cell damage. We tested this prediction by determining whether infected Schwann cells or Schwann cells transfected with the PDNF-encoding plasmid resisted the toxic action of H₂O₂, which causes oxidative stress and caspase-mediated apoptosis in various cell types, including neurons (27, 28). In a terminal deoxynucleotidyl transferase-mediated dUTP nick end labeling (TUNEL) assay, uninfected (Sc) or T. cruzi-infected (Sc-Inf) cells were treated with 100 μM H₂O₂ for 6 hours, fixed, and assessed for apoptosis by the TUNEL assay. Cells were counterstained with DAPI to reveal cell density. We found that, after 6 hours, 100 μM H₂O₂ induced apoptosis in ˜25% of uninfected Schwann cells, and, after 24 hours, only half of the cells survived (FIG. 6a). In contrast to uninfected cells, T. cruzi-bearing cells were protected against 100 μM H₂O₂-induced cellular degeneration (FIG. 6a).

T. cruzi-infected Schwann cells were also found to be resistant to high concentration of H₂O₂, even at 500 μM H₂O₂. In one experiment, uninfected (Sc) or T. cruzi-infected (Sc-Inf) cells were treated with 500 μM of H₂O₂ for 24 hours, fixed, and assessed for apoptosis by the TUNEL fluorescent assay. Cells were counterstained with DAPI and T. cruzi polyclonal antibody. The fluorescent assay showed that when almost 90% of uninfected cells displayed apoptosis-related DNA damage, few of the T. cruzi-bearing cells showed signs of apoptosis, although parasites seemed to be destroyed, possibly because they are catalase-deficient and unable to neutralize H₂O₂ (29). Resistance of infected cells to oxidative stress depended on the kinase activity of Akt because it was lost in cells are treated with Akti VIII (FIG. 6a). Similar to the infected cells (FIG. 6a), Schwann cells transfected with a plasmid encoding PDNF were protected from H₂O₂-induced oxidative stress, and this survival-promoting activity was abrogated after treatment with Akti VIII (FIG. 6b).

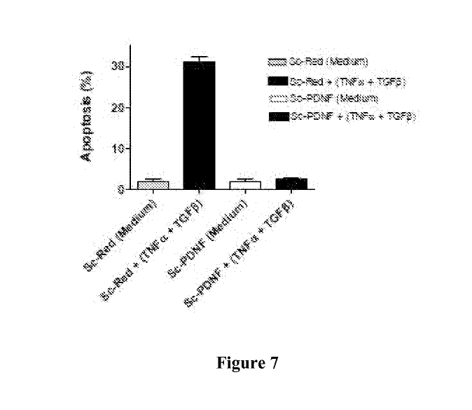

To determine whether the oxidative stress-resistant phenotype extended to another apoptotic stimulus relevant to T. cruzi infection in vivo, we assessed whether intracellular PDNF protected human Schwann cells against an immune-based cell death mechanism. The proinflammatory cytokine TNF-α kills Schwann cells synergistically with transforming growth factor-β (TGF-β), an event likely related to peripheral nervous system disorders (30). In addition, TNF-α appears to mediate the killing of human Schwann cells by cytotoxic T cells (31). We therefore ascertained whether the introduction of PDNF into the Schwann cell cytosol neutralized the toxicity caused by TNF-α and TGF-β. Based on the TUNEL assay, apoptosis in control Schwann cells increased ˜14-fold in medium containing TNF-α (20 ng/ml) and TGF-β (40 ng/ml) (FIG. 7). Introducing PDNF into the Schwann cell cytosol rescued cells from death caused by TNF-α and TGF-β, because only 2.5%±0.7 of the cells underwent apoptosis in the presence of the two cytokines (FIG. 7). The anti-apoptotic effect of intracellular PDNF was abolished by the Akt inhibitor Akti VIII (FIG. 7), suggesting that it depended on the activation of Akt by PDNF.

4. Discussion

Akt1, Akt2, and Akt3, originally identified as the transforming oncogenes of a murine retrovirus (32), are critical mediators of signal transduction pathways downstream of activated receptor tyrosine kinases (RTKs) and PI3K. Activated Akt promotes cell survival by inhibiting the function of pro-apoptotic proteins, particularly Bc1-2 homology domain 3 (BH3)-only proteins such as BAD. Once phosphorylated by Aid, BAD binds to the scaffolding adaptor protein 14-3-3, which prevents the release of cytochrome c from mitochondria (33). In addition, Akt inhibits the expression of genes encoding BH3-only proteins, such as the pro-apoptotic cytokine Fas ligand, by phosphorylating and inactivating transcription factors such as FOXO (34, 35). Akt directly interferes with the caspase cascade by phosphorylating procaspase-9 and rendering it inactive, thereby inhibiting the activation of effector caspases (36). Activated Akt also promotes cell survival through crosstalk with other signaling cascades such as those involving nuclear factor KB (NF-κB) (37) and the mitogen-activated protein kinases (MAPKs) c-Jun N-terminal kinase (INK) and p38 (38). Finally, the activation of Akt indirectly supports cell survival by increasing uptake of nutrients, metabolism, and maintenance of mitochondrial membrane potential (39).

Here, we demonstrated that Akt interacted with T. cruzi PDNF, a neuraminidase and trans-sialidase, when either the parasite or recombinant PDNF was in the cytosol. The clue for identifying this interaction between T. cruzi and Akt was the bioinformatics Scansite program (21), which predicted five target sites for phosphorylation by Akt in the N-terminal region of PDNF (FIG. 1A). These sites are located in β-hairpin loops on the surface of PDNF (FIG. 1A) and, thus, are readily accessible for phosphorylation by Akt. Hairpin loops or reverse turns commonly mediate specific molecular interactions such as ligand-receptor and antibody-antigen binding (40).

Phosphorylation of PDNF was determined by a phosphorylation site readout that depended on specific antibodies that recognized Akt-phosphorylated substrates, an extremely valuable tool used in the past few years to identify and characterize new substrates of Akt (10). Phosphorylation of PDNF by Akt was also demonstrated by an in vitro kinase assay. Based on colocalization studies with antibodies specific for PDNF and for Akt-dependent phospho-peptides, most substrates phosphorylated by Akt in T. cruzi-infected Schwann cells colocalized with PDNF. However, PDNF may not be the only T. cruzi protein that is targeted for phosphorylation by Aid, as further analysis of T. cruzi proteomics with the Scansite program revealed at least 8 additional potential substrates of Akt, including Tc85-11, a member of the PDNF-trans-sialidase superfamily thought to mediate T. cruzi-host cell interactions (41).

Akt phosphorylates PDNF of intracellular T. cruzi predominantly late in the infection cycle (FIG. 3B), when the parasite burden is large. The generation of phosphorylated PDNF correlated with the enhanced activation of Akt. Phosphorylation of PDNF and enhancement of the activation of Akt was reproduced by transfecting Schwann cells with a plasmid encoding PDNF (FIG. 4A). In addition, intracellular PDNF increased the expression of the gene encoding Akt and inhibited transcription of at least three genes that encode pro-apoptotic factors (caspase-9, the Bc12-family member BAX, and the transcription factor FOXO) (FIG. 5). These biochemical, enzymatic, and genetic alterations in T. cruzi-infected and PDNF-transfected cells would likely endow the cells with several properties that promote host cell viability such as the resistance to apoptotic stimuli that we have demonstrated here (FIGS. 6 and 7).

How could intracellular PDNF activate Akt? Normally, PI3K-Akt signaling is activated when PI3K, which resides in the cytoplasm, binds, through its regulatory p85 subunit, to either an RTK at the cell surface or to activated adaptor molecules. As a result, PI3K localizes to the plasma membrane, where it phosphorylates the membrane lipid phosphatidylinositol 4,5-bisphosphate (PIP₂) to produce phosphatidylinositol 3,4,5-trisphosphate (PIP₃). This leads to the activation of downstream signaling pathways that control cell growth and survival. Integrated cascades of phosphorylation of tyrosine and serine or threonine residues play an essential role in transducing signals through the PI3K-Akt pathway. The mechanism by which this occurs is through the pTyr-binding domains Src homology 2 (SH2) and phosphotyrosine-binding (PTB) (42), and, although less well-studied, through pSer- and pThr-binding motifs (43). Phosphorylation on serine or threonine residues, initially discovered as a way to allosterically regulate catalytic activity, can also create sites for pSer- or pThr-binding signaling molecules, which result in their recruitment to signaling complexes. Such molecules currently include 14-3-3 proteins, WW domains, forkhead-associated regions, WD40 repeats, and Polo box domains (43, 44). In particular 14-3-3 proteins are implicated in the regulation of cell cycle, apoptosis, and activation of the Raf-MAPK pathway (43) and in PI3K-Akt signaling and cell survival (45). Proteins that bind to motifs that contain pSer or pThr sites generally recognize sequences that overlap with sites phosphorylated by Akt (46) and thus can potentially complex with PDNF through its pSer- and pThr-containing motifs.

On the other hand, the C-terminal proline-rich region (PRR) of PDNF contains multiple PxxP (SEQ ID NO: 16) repeats (P, proline; x, any aliphatic residue) (FIG. 1A) which suggest a capability to interact with signaling proteins that contain SH3 domains, such as Src and PI3K (47). For example, the poly-proline motif PxxP of NS1, an influenza A virus protein, is essential for its binding to the p85β subunit of PI3K and the activation of the PI3K-Akt pathway in response to viral infection (48). So the possibility exists that PDNF is directly integrated into the assembly of the PI3K signalosome by forming a scaffold between the SH3 domain of PI3K and the pSer- and pThr-binding motif(s) of 14-3-3, which could mediate the formation of a complex through the SH2 domain of Shc with an RTK and promote the activation of Akt (45).

The biological functions of PDNF are mostly studied in the context of its sialic acid-binding properties (5, 49), largely because these studies are focused on the neuraminidase (4) and sialyl-transferase (26, 50, 51) activities of the protein. However, PDNF has also intrinsic neurotrophic properties (11) that result from its binding to the neurotrophin receptors TrkA and TrkC (6, 7), which do not require its neuraminidase or trans-sialidase activities (11, 52). The interaction of T. cruzi with TrkA drives the invasion of neuronal and non-neuronal cells (12). Also independent of trans-sialidase activity is the promotion of the survival of endothelial cells through as yet unknown receptors (53).

The neuraminidase and trans-sialidase activities of PDNF require substrates (sialyl and galactosyl residues) that are available only in the extracellular environment. These extracellular actions of T. cruzi PDNF are short-lived as they occur when T. cruzi is establishing its long-term intracellular habitat. Therefore, it was most surprising to find that PDNF interacted with a signaling protein (Akt) located in the cell cytosol where sialo-glycoconjugates, the substrates of neuraminidase-trans-sialidase, are absent.

In sum, the results presented here are consistent with T. cruzi, through PDNF, interacting with and activating Akt signaling while residing in the cytosol of infected cells. Activation was sustained (lasted days) and was most prominent late in the infection cycle when parasite burden was maximal, a time when the intracellular niche of T. cruzi needs the most protection. In addition, T. cruzi activates Aid, albeit transiently, at the port-of-entry of the cell habitat, when it binds to the NGF receptor TrkA to invade cells (9, 11). Collectively, these findings characterize a parasite effector protein with a unique dual activity to modulate host signaling responses during infection, and present a previously undescribed paradigm for the interaction of a pathogen with its host.

### Example 2

### Materials and Methods

1. Materials

Dulbecco's modified Eagle's medium (DMEM), penicillin/streptomycin stock, fetal calf serum (FCS), and G418 were from GIBCO; DAPI (4′,6-diamidino-2-phenylindole) and LY294002 were from Sigma and the Aid inhibitor Akti VIII was from EMD Chemicals. Antibodies against pAkt (Ser⁴⁷³ and Thr³⁰⁸), Akt, and phosphorylated substrates of Akt were from Cell Signaling Technology. The PDNF-specific mAB TCN-2 (T cruzi neuraminidase monoclonal antibody-2) was isolated as described earlier (23). Alexa-conjugated anti-mouse and anti-rabbit secondary antibodies were from Molecular Probes and the horse radish peroxidase (HRP)-conjugated secondary antibody was from Chemicon. The ECL kit was purchased from PerkinElmer. The antiprotease cocktail was from Roche Molecular Biochemicals. Recombinant, full-length PDNF cDNA (clone 19Y) was expressed in E. coli and purified by affinity chromatography, and the trans-sialidase activity assay was performed as described before (26).

2. Cell Culture and Infections with T. cruzi

Immortalized human Schwann cells (54) were maintained in DMEM supplemented with 10% FCS and penicillin/streptomycin at 37° C. in 5% CO₂. Infections were performed with T. cruzi trypomastigotes (Silvio strain) at 2×10⁵ parasites/ml or at a parasite:cell ratio of 50:1. After 2 or 3 hours, monolayers were washed to remove unattached parasites and cells were then maintained in medium containing 2% FCS for 3 to 5 days to complete the intracellular infection cycle.

3. Cloning of TCNA and Transfections

The coding region of the gene encoding PDNF (formerly T. cruzi neuraminidase, or TCNA) was amplified from the TS19y clone (20) with the primers TCNA-F: 5′-CCGCTCGAGATGGGTTTGGCACCCGGATCG-3′ (SEQ ID NO: 11) and TCNA-R: 5′-TCCCCGCGGTCAGAAAACTGCCATAAA (SEQ ID NO: 12) (restriction sites for XhoI and SacII are underlined) and inserted into the pIRES2-DsRed-Express vector (Clontech). Selected recombinant plasmids were purified with the Qiagen kit, sequenced at Tufts University Core facility, and used for transfections. Cells were plated at 1×10⁵ cells/ml in 100-mm plates 20 hours before their transfection with 10 μg of total DNA per plate with the FuGENE HD transfection reagent (Roche Diagnostics) as recommended by the manufacturer. Transfected cells were selected with G418 (1 mg/ml) and analyzed for the presence of PDNF by Western blotting and trans-sialidase activity assays (26). Schwann cells transfected with the plasmid encoding PDNF are named Sc-PDNF cells, whereas Schwann cells transfected with empty vector are named Sc-Red cells.

4. Immunoprecipitations and Western Blotting

Cell monolayers were lysed with lysis buffer (20 mM tris-HCl (pH 7.5), 150 mM NaCl, 1 mM EDTA, 1% Triton X-100, 2.5 mM sodium pyrophosphate, 1 mM glycerophosphate, 1 mM Na₃VO₄, 1 μg/mL leupeptin and 1 mM phenylmethylsulfonyl fluoride (PMSF)) on ice for 10 min, cleared by centrifugation (12,000×g for 10 min at 4° C.) and immunoprecipitated with an antibody specific for Akt-phosphorylated substrate or with TCN-2 at 4° C. overnight Immunoprecipitates were collected with protein G-Sepharose for 2 hours at 4° C., washed with lysis buffer, and resuspended in SDS-sample buffer for SDS-PAGE and Western blotting analysis. In other experiments, total cell lysates (30 to 50 μg of protein) were resolved by SDS-PAGE, transferred to nitrocellulose membranes, and incubated with the relevant primary antibodies, followed by incubation with HRP-conjugated secondary antibody and visualization by ECL.

5. Immunocytochemistry

Monolayers of infected or transfected cells were fixed with 4% paraformaldehyde for 30 min at 4° C., permeabilized in 0.2% Triton X-100, and blocked in 10% goat serum, overnight at 4° C. Cells were incubated with the appropriate primary antibody in 5% goat serum overnight at 4° C., washed in phosphate-buffered saline (PBS) and incubated with Alexa Fluor 488-conjugated anti-rabbit and Alexa Fluor 568-conjugated anti-mouse secondary antibodies (Molecular Probes). Cell nuclei were visualized with DAPI (250 ng/ml). Images were analyzed by fluorescence microscopy with Spot camera software (Diagnostic Instruments).

6. In Vitro Kinase Assays

Akt was purified from lysates of serum-stimulated Schwann cells with an antibody specific for Akt and protein G-Sepharose. Isolated complexes were resuspended in kinase buffer (25 mM tris-HCl (pH 7.5), 5, 5 mM (3-glycerophosphate, 2 mM dithiothreitol (DTT), 0.1 mM Na₃VO₄, 10 mM MgCl₂), supplemented with 1 mM adenosine triphosphate (ATP) and PDNF isolated from a bacterial expression system (bPDNF). After 30 min at 30° C., the kinase reaction was terminated with SDS-Laemmli sample buffer, and samples were analyzed by Western blotting by incubation with an antibody against Akt-phosphorylated substrates. PDNF was identified by comparison with a Coomassie blue-stained sample of bPDNF run in parallel with the samples for Western blotting.

7. Apoptosis Assays

Infected Schwann cells, Sc-Red cells, and Sc-PDNF cells were treated for 24 hours with 100 to 500 μM H₂O₂ with or without pretreatment with the Akt inhibitor Akti VIII (10 μM), fixed, and visualized by fluorescence microscopy after staining with DAPI and TUNEL reagents with the In Situ Cell Death Detection kit (Roche). Cell survival was measured by MTT-based CellTiter kit (Promega). Alternatively, Sc-Red and Sc-PDNF cells were grown for 4 days in DMEM, 1% bovine serum albumin (BSA) with or without TNF-α (20 ng/ml) and TGF-β1 (40 ng/ml) and with or without Akti VIII (10 μM).

8. cDNA Array Hybridizations

These analyses were performed as described previously (55, 56). The concentration and quality of total RNA isolated from Sc-Red cells and Sc-PDNF cells were estimated by spectrophotometry (A₂₆₀ nm/A₂₈₀ nm of 1.9 to 2.1) and by agarose gel electrophoresis. Hybridization and data analysis were performed by the Tufts Expression Array Core with cDNAs prepared from Sc-Red cells and Sc-PDNF cells labeled with aminoallyl (aa)-dUTP Cy3 or Cy5 dyes, respectively. The two differently dye-labeled cDNAs were hybridized with the same microarray slide containing 48,500 human genes (Microarrays Inc) for 16 hours. After washing, the slide was scanned at 550 nm and 649 nm for Cy-3 and Cy-5 dyes, respectively, in a ScanArray 4000 scanner (PerkinElmer). Images were overlaid and analyzed with QuantArray spot quantification software (Packard BioChip Technologies). The ratio of both fluorescence intensities for each spot reflected the ratio of each gene expressed in the control and treatment samples. Local background values were subtracted from the spot intensities and filtered based on three standard deviations above background. For each gene, ratios of red (Cy-5) over green (Cy-3) intensities (I) were calculated and normalized through a Lowess Fit of the log₂ ratios (log₂(Icy-5/Icy-3)) over the log₂ of the total intensity (log₂(Icy-5. Icy-3)). Mean ratios were calculated from the duplicate spots, and only values with a covariance (CV) b0.5 were further taken into account. Normalized ratios that were statistically significant with a two-tailed t test (5% level) between the dye-swap repeat and higher than 1 or lower than −1 (log₂ scale) were considered differentially expressed.

9. Quantitative Real-Time PCR (qPCR)

These assays were performed as described previously (57, 58). In brief, RNA was isolated with the Trizol reagent and chloroform, cDNA was synthesized from 500 ng of total RNA with Superscript III reverse transcriptase primed with random hexamers, and quantitative PCR reactions were performed with QuantiTect SYBR Green PCR kit (Qiagen) on an Applied Biosystems 7300 Real-Time PCR system. Conditions for the PCR reactions were: 95° C. for 15 min, followed by 50 cycles at 94° C. for 15 s, 54° C. for 30 s, and 72° C. for 45 s, concluding with a dissociation stage to measure amplification product specificity. Primers used to calculate Akt expression were synthesized as described previously (57). Experiments were performed in triplicate, and fold differences were calculated by the 2^{-ΔΔCt} method (ΔΔCt=(Ct_{target gene}−Ct_{b-actin})_{treated}−(Ct_{target gene}−Ct_{b-actin})_{untreated}) as described previously (58).

10. Data Analysis

The results from the experiments were expressed as the means±the standard error of the mean (SEM). Statistical difference was evaluated with the unpaired t test.

### REFERENCES

1 L. O. Andrade, N. W. Andrews, The Trypanosoma cruzi-host-cell interplay: location, invasion, retention. Nat Rev Microbiol 3, 819-823 (2005)
2 H. B. Tanowitz, L. V. Kirchhoff, D. Simon, S. A. Morris, L. M. Weiss, M. Wittner, Chagas' disease. Clin Microbiol Rev 5, 400-419 (1992)
3 T. P. Combs, Nagajyothi, S. Mukherjee, C. J. de Almeida, L. A. Jelicks, W. Schubert, Y. Lin, D. S. Jayabalan, D. Zhao, V. L. Braunstein, S. Landskroner-Eiger, A. Cordero, S. M. Factor, L. M. Weiss, M. P. Lisanti, H. B. Tanowitz, P. E. Scherer, The adipocyte as an important target cell for Trypanosoma cruzi infection. J Biol Chem 280, 24085-24094 (2005)
4 M. E. Pereira, A developmentally regulated neuraminidase activity in Trypanosoma cruzi. Science 219, 1444-1446 (1983)
5 S. Schenkman, D. Eichinger, M. E. Pereira, V. Nussenzweig, Structural and functional properties of Trypanosoma trans-sialidase. Annu Rev Microbiol 48, 499-523 (1994)
6 M. V. Chuenkova, M. Pereiraperrin, Chagas' disease parasite promotes neuron survival and differentiation through TrkA nerve growth factor receptor. J Neurochem 91, 385-394 (2004)
7 C. Weinkauf, M. Pereiraperrin, Trypanosoma cruzi promotes neuronal and glial cell survival through the neurotrophic receptor TrkC. Infect Immun (2009)
8 E. J. Huang, L. F. Reichardt, Trk receptors: roles in neuronal signal transduction. Annu Rev Biochem 72, 609-642 (2003)
9 A. Bellacosa, C. C. Kumar, A. Di Cristofano, J. R. Testa, Activation of AKT kinases in cancer: implications for therapeutic targeting. Adv Cancer Res 94, 29-86 (2005)
10 B. D. Manning, L. C. Cantley, AKT/PKB signaling: navigating downstream. Cell 129, 1261-1274 (2007)
11 M. V. Chuenkova, M. A. Pereira, A trypanosomal protein synergizes with the cytokines ciliary neurotrophic factor and leukemia inhibitory factor to prevent apoptosis of neuronal cells. Mol Biol Cell 11, 1487-1498 (2000)
12 M. de Melo-Jorge, M. PereiraPerrin, The Chagas' disease parasite Trypanosoma cruzi exploits nerve growth factor receptor TrkA to infect mammalian hosts. Cell Host Microbe 1, 251-261 (2007)
13 D. Golgher, R. T. Gazzinelli, Innate and acquired immunity in the pathogenesis of Chagas disease. Autoimmunity 37, 399-409 (2004)
14 I. Rosenberg, R. P. Prioli, E. Ortega-Barria, M. E. Pereira, Stage-specific phospholipase C-mediated release of Trypanosoma cruzi neuraminidase. Mol Biochem Parasitol 46, 303-305 (1991)
15 U. Frevert, S. Schenkman, V. Nussenzweig, Stage-specific expression and intracellular shedding of the cell surface trans-sialidase of Trypanosoma cruzi. Infect Immun 60, 2349-2360 (1992)
16 I. A. Rosenberg, R. P. Prioli, J. S. Mejia, M. E. Pereira, Differential expression of Trypanosoma cruzi neuraminidase in intra- and extracellular trypomastigotes. Infect Immun 59, 464-466 (1991)
17 R. P. Prioli, J. S. Mejia, T. Aji, M. Aikawa, M. E. Pereira, Trypanosoma cruzi: localization of neuraminidase on the surface of trypomastigotes. Trop Med Parasitol 42, 146-150 (1991)
18 D. R. Alessi, F. B. Caudwell, M. Andjelkovic, B. A. Hemmings, P. Cohen, Molecular basis for the substrate specificity of protein kinase B; comparison with MAPKAP kinase-1 and p70 S6 kinase. FEBS Lett 399, 333-338 (1996)
19 M. E. Pereira, J. S. Mejia, E. Ortega-Barria, D. Matzilevich, R. P. Prioli, The Trypanosoma cruzi neuraminidase contains sequences similar to bacterial neuraminidases, YWTD repeats of the low density lipoprotein receptor, and type III modules of fibronectin. J Exp Med 174, 179-191 (1991)
20 M. Chuenkova, M. Pereira, G. Taylor, trans-sialidase of Trypanosoma cruzi: location of galactose-binding site(s). Biochem Biophys Res Commun 262, 549-556 (1999)
21 M. B. Yaffe, G. G. Leparc, J. Lai, T. Obata, S. Volinia, L. C. Cantley, A motif-based profile scanning approach for genome-wide prediction of signaling pathways. Nat Biotechnol 19, 348-353 (2001)
22 M. F. Amaya, A. G. Watts, I. Damager, A. Wehenkel, T. Nguyen, A. Buschiazzo, G. Paris, A. C. Frasch, S. G. Withers, P. M. Alzari, Structural insights into the catalytic mechanism of Trypanosoma cruzi trans-sialidase. Structure 12, 775-784 (2004)
23 R. P. Prioli, J. S. Mejia, M. E. Pereira, Monoclonal antibodies against Trypanosoma cruzi neuraminidase reveal enzyme polymorphism, recognize a subset of trypomastigotes, and enhance infection in vitro. J Immunol 144, 4384-4391 (1990)
24 B. D. Manning, A. R. Tee, M. N. Logsdon, J. Blenis, L. C. Cantley, Identification of the tuberous sclerosis complex-2 tumor suppressor gene product tuberin as a target of the phosphoinositide 3-kinase/akt pathway. Mol Cell 10, 151-162 (2002)
25 V. Calleja, M. Laguerre, P. J. Parker, B. Larijani, Role of a novel PH-kinase domain interface in PKB/Akt regulation: structural mechanism for allosteric inhibition. PLoS Biol 7, e17 (2009)
26 P. Scudder, J. P. Doom, M. Chuenkova, I. D. Manger, M. E. Pereira, Enzymatic characterization of beta-D-galactoside alpha 2,3-trans-sialidase from Trypanosoma cruzi. J Biol Chem 268, 9886-9891 (1993)
27 V. D. Nair, C. W. Olanow, Differential modulation of Akt/glycogen synthase kinase-3beta pathway regulates apoptotic and cytoprotective signaling responses. J Biol Chem 283, 15469-15478 (2008)
28 K. Shenoy, Y. Wu, S. Pervaiz, LY303511 enhances TRAIL sensitivity of SHEP-1 neuroblastoma cells via hydrogen peroxide-mediated mitogen-activated protein kinase activation and up-regulation of death receptors. Cancer Res 69, 1941-1950 (2009)
29 R. Docampo, S. N. Moreno, Free radical metabolites in the mode of action of chemotherapeutic agents and phagocytic cells on Trypanosoma cruzi. Rev Infect Dis 6, 223-238 (1984)
30 A. M. Skoff, R. P. Lisak, B. Bealmear, J. A. Benjamins, TNF-alpha and TGF-beta act synergistically to kill Schwann cells. J Neurosci Res 53, 747-756 (1998)
31 B. Bonetti, P. Valdo, G. Ossi, L. De Toni, B. Masotto, S. Marconi, N. Rizzuto, E. Nardelli, G. Moretto, T-cell cytotoxicity of human Schwann cells: TNFalpha promotes fasL-mediated apoptosis and IFN gamma perforin-mediated lysis. Glia 43, 141-148 (2003)
32 A. Bellacosa, J. R. Testa, S. P. Staal, P. N. Tsichlis, A retroviral oncogene, akt, encoding a serine-threonine kinase containing an SH2-like region. Science 254, 274-277 (1991)
33 S. R. Datta, A. Katsov, L. Hu, A. Petros, S. W. Fesik, M. B. Yaffe, M. E. Greenberg, 14-3-3 proteins and survival kinases cooperate to inactivate BAD by BH3 domain phosphorylation. Mol Cell 6, 41-51 (2000)
34 H. Tran, A. Brunet, E. C. Griffith, M. E. Greenberg, The many forks in FOXO's road. Sci STKE 2003, RES (2003)
35 A. Brunet, A. Bonni, M. J. Zigmond, M. Z. Lin, P. Juo, L. S. Hu, M. J. Anderson, K. C. Arden, J. Blenis, M. E. Greenberg, Akt promotes cell survival by phosphorylating and inhibiting a Forkhead transcription factor. Cell 96, 857-868 (1999)
36 M. H. Cardone, N. Roy, H. R. Stennicke, G. S. Salvesen, T. F. Franke, E. Stanbridge, S. Frisch, J. C. Reed, Regulation of cell death protease caspase-9 by phosphorylation. Science 282, 1318-1321 (1998)
37 J. A. Romashkova, S. S. Makarov, NF-kappaB is a target of AKT in anti-apoptotic PDGF signalling. Nature 401, 86-90 (1999)
38 A. H. Kim, T. Sasaki, M. V. Chao, JNK-interacting protein 1 promotes Akt1 activation. J Biol Chem 278, 29830-29836 (2003)
39 D. R. Plas, C. B. Thompson, Akt-dependent transformation: there is more to growth than just surviving. Oncogene 24, 7435-7442 (2005)
40 R. M. Hughes, M. L. Waters, Model systems for beta-hairpins and beta-sheets. Curr Opin Struct Biol 16, 514-524 (2006)
41 M. Marroquin-Quelopana, S. Oyama, Jr., T. Aguiar Pertinhez, A. Spisni, M. Aparecida Juliano, L. Juliano, W. Colli, M. J. Alves, Modeling the Trypanosoma cruzi Tc85-11 protein and mapping the laminin-binding site. Biochem Biophys Res Commun 325, 612-618 (2004)
42 M. B. Yaffe, Phosphotyrosine-binding domains in signal transduction. Nat Rev Mol Cell Biol 3, 177-186 (2002)
43 M. B. Yaffe, A. E. Elia, Phosphoserine/threonine-binding domains. Curr Opin Cell Biol 13, 131-138 (2001)
44 D. P. Seeburg, D. Pak, M. Sheng, Polo-like kinases in the nervous system. Oncogene 24, 292-298 (2005)
45 E. F. Barry, F. A. Felquer, J. A. Powell, L. Biggs, F. C. Stomski, A. Urbani, P. Hoffmann, M. C. Wilce, M. A. Grimbaldeston, A. F. Lopez, M. A. Guthridge, 14-3-3:SHC scaffolds integrate phoshoserine and phosphotyrosine signaling to regulate PI 3-kinase activation and cell survival. J Biol Chem (2009)
46 M. Y. Liu, S. Cai, A. Espejo, M. T. Bedford, C. L. Walker, 14-3-3 interacts with the tumor suppressor tuberin at Akt phosphorylation site(s). Cancer Res 62, 6475-6480 (2002)
47 T. Kaneko, L. Li, S. S. Li, The SH3 domain—a family of versatile peptide- and protein-recognition module. Front Biosci 13, 4938-4952 (2008)
48 Y. K. Shin, Y. Li, Q. Liu, D. H. Anderson, L. A. Babiuk, Y. Zhou, SH3 binding motif 1 in influenza A virus NS1 protein is essential for PI3K/Akt signaling pathway activation. J Virol 81, 12730-12739 (2007)
49 C. A. Buscaglia, V. A. Campo, A. C. Frasch, J. M. Di Noia, Trypanosoma cruzi surface mucins: host-dependent coat diversity. Nat Rev Microbiol 4, 229-236 (2006)
50 A. Buschiazzo, G. A. Tavares, O. Campetella, S. Spinelli, M. L. Cremona, G. Paris, M. F. Amaya, A. C. Frasch, P. M. Alzari, Structural basis of sialyltransferase activity in trypanosomal sialidases. EMBO J19, 16-24 (2000)
51 S. Schenkman, L. Pontes de Carvalho, V. Nussenzweig, Trypanosoma cruzi trans-sialidase and neuraminidase activities can be mediated by the same enzymes. J Exp Med 175, 567-575 (1992)
52 M. V. Chuenkova, M. PereiraPerrin, A synthetic peptide modeled on PDNF, Chagas' disease parasite neurotrophic factor, promotes survival and differentiation of neuronal cells through TrkA receptor. Biochemistry 44, 15685-15694 (2005)
53 W. B. Dias, F. D. Fajardo, A. V. Graca-Souza, L. Freire-de-Lima, F. Vieira, M. F. Girard, B. Bouteille, J. O. Previato, L. Mendonca-Previato, A. R. Todeschini, Endothelial cell signalling induced by trans-sialidase from Trypanosoma cruzi. Cell Microbiol 10, 88-99 (2008)
54 M. V. Chuenkova, F. B. Furnari, W. K. Cavenee, M. A. Pereira, Trypanosoma cruzi trans-sialidase: a potent and specific survival factor for human Schwann cells by means of phosphatidylinositol 3-kinase/Akt signaling. Proc Natl Acad Sci USA 98, 9936-9941 (2001)
55 A. A. Alizadeh, M. B. Eisen, R. E. Davis, C. Ma, I. S. Lossos, A. Rosenwald, J. C. Boldrick, H. Sabet, T. Tran, X. Yu, J. I. Powell, L. Yang, G. E. Marti, T. Moore, J. Hudson, Jr., L. Lu, D. B. Lewis, R. Tibshirani, G. Sherlock, W. C. Chan, T. C. Greiner, D. D. Weisenburger, J. O. Armitage, R. Warnke, R. Levy, W. Wilson, M. R. Grever, J. C. Byrd, D. Botstein, P. O. Brown, L. M. Staudt, Distinct types of diffuse large B-cell lymphoma identified by gene expression profiling. Nature 403, 503-511 (2000)
56 I. J. Blader, I. D. Manger, J. C. Boothroyd, Microarray analysis reveals previously unknown changes in Toxoplasma gondii-infected human cells. J Biol Chem 276, 24223-24231 (2001)
57 V. Gagnon, I. Mathieu, E. Sexton, K. Leblanc, E. Asselin, AKT involvement in cisplatin chemoresistance of human uterine cancer cells. Gynecol Oncol 94, 785-795 (2004)
58 K. J. Livak, T. D. Schmittgen, Analysis of relative gene expression data using real-time quantitative PCR and the 2(-Delta Delta C(T)) Method. Methods 25, 402-408 (2001)
59 A. Buschiazzo, M. F. Amaya, M. L. Cremona, A. C. Frasch, P. M. Alzari, The crystal structure and mode of action of trans-sialidase, a key enzyme in Trypanosoma cruzi pathogenesis. Mol Cell 10, 757-768 (2002)

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents and NCBI Entrez or gene ID sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. A method of reducing cell apoptosis, comprising:
delivering to a cell a nucleic acid molecule comprising a nucleotide sequence that encodes parasite-derived neurotrophic factor (PDNF), or a fragment of PDNF, wherein the PDNF or PDNF fragment binds to Akt kinase.

2. The method of claim 1, wherein the nucleic acid molecule is administered to a mammalian subject in need of reducing cell apoptosis.

3. A method of activating Akt kinase in a cell, comprising
delivering to the cell a nucleic acid molecule comprising a nucleotide sequence that encodes parasite-derived neurotrophic factor (PDNF), or a fragment of PDNF, wherein the PDNF or PDNF fragment binds to Akt kinase.

4. The method of claim 3, wherein the nucleic acid molecule is administered to a mammalian subject in need of reducing cell apoptosis.

5. A method of reducing the effect of an apoptotic-inducing agent on a mammalian subject, comprising:
administering to the subject in need thereof a nucleic acid molecule comprising a nucleotide sequence that encodes PDNF, or a fragment of PDNF, wherein the PDNF or PDNF fragment binds to Akt kinase.

6. The method of claim 5, wherein the apoptotic-inducing agent is a cytokine.

7. The method of claim 6, wherein the cytokine is TGF-β or TNF-α.

8. The method of claim 5, wherein the apoptotic-inducing agent causes oxidative stress.

9. The method of claim 8, wherein the apoptotic-inducing agent produces H₂O₂ or free radicals.

10. A method of treating a condition in a mammalian subject wherein the condition is alleviated by an increased activity of Akt kinase, comprising:
delivering to the cell a nucleic acid molecule comprising a nucleotide sequence that encodes PDNF, or a fragment of PDNF, wherein the PDNF or PDNF fragment binds to Akt kinase.

11. The method of claim 5, wherein the mammalian subject is a human.

12. The method of claim 5, wherein the mammalian subject is in need of reducing apoptosis of neurons or glial cells.

13. The method of claim 12, wherein the glial cells are Schwann cells.

14. The method of claim 5, wherein the subject is suffering from or susceptible to a neurodegenerative disease.

15. The method of claim 1, wherein the PDNF or PDNF fragment does not comprise a secretory signal peptide sequence.

16. The method of claim 1, wherein the PDNF or PDNF fragment comprises an Akt phosphorylation site.

17. The method of claim 16, wherein the phosphorylation of PDNF or PDNF fragment induces an increase in Akt kinase activity.

18. The method of claim 16, wherein the phosphorylation of PDNF or PDNF fragment induces an increase in the expression level of a gene that encodes Akt kinase.

19. The method of claim 16, wherein the phosphorylation of PDNF or PDNF fragment induces an decrease in the activity of a pro-apoptotic protein, or an decrease in the expression level of a gene encoding a pro-apoptotic protein.

20. The method of claim 19, wherein the pro-apoptotic protein is Caspase-9, FOXO, or BAX.

21. The method of claim 1, wherein the PDNF comprises an amino acid sequence that is selected from the group consisting of (a) the amino acid sequence set forth in SEQ ID NO: 5; (b) the amino acid sequence set forth in residues 1 to 588 of SEQ ID NO: 4; and (c) an amino acid sequence that is at least 85% identical to (a) or (b).

22. The method of claim 16, wherein the phosphorylation site of the PDNF or PDNF fragment is a serine or threonine residue that corresponds to positions S91, T17, T304, T597, or S123 of SEQ ID NO:5.

23. The method of claim 16, wherein the phosphorylation site of the PDNF or PDNF fragment is S91, T17, T304, T597, or S123 of SEQ ID NO:5.

24. The method of claim 1, wherein the nucleic acid molecule comprises a nucleotide sequence that is selected from the group consisting of: (a) nucleotides 234 to 2123 of the nucleotide sequence set forth in SEQ ID NO: 1; (b) nucleotides 484 to 2248 of the nucleotide sequence set forth in SEQ ID NO: 3; and (c) a nucleotide sequence that is at least 85% identical to (a) or (b).

25. The method of claim 1, wherein the nucleic acid molecule is a vector derived from an adenovirus, an adeno-associated virus, a lentivirus, or an alphavirus.

26. The method of claim 1, wherein the nucleic acid molecule is a replication-deficient viral vector.

27. The method of claim 1, wherein the nucleic acid molecule is a vector comprising a nucleotide sequence that encodes the PDNF or PDNF fragment that is operably linked to an expression control sequence that promotes the expression of the PDNF or PDNF fragment in a mammalian cell.

28. The method of claim 27, wherein the expression control sequence is a promoter, an enhancer, a ribosome entry site, or a polyadenylation sequence.
